(19) United States
(12) Patent Application Publication
(10) Pub. No.: US 2024/0177861 A1
(43) Pub. Date: May 30, 2024

(54) IMAGE DIAGNOSTIC SYSTEM AND IMAGE DIAGNOSTIC METHOD

(71) Applicant: SONY GROUP CORPORATION, TOKYO (JP)

(72) Inventors: KENJI YAMANE, TOKYO (JP); KENJI SUZUKI, TOKYO (JP); TOYA TERAMOTO, TOKYO (JP); YUKI ONO, TOKYO (JP); YOSHIYUKI KOBAYASHI, TOKYO (JP); MASATO ISHII, TOKYO (JP)

(21) Appl. No.: 18/550,315

(22) PCT Filed: Dec. 30, 2021
(86) PCT No.: PCT/JP2021/049032

§ 371 Date: Sep. 13, 2023

(30) Foreign Application Priority Data
Mar. 22, 2021 (JP) 2021-047996

(51) Int. Cl. G16H 50/20; G06T 7/00; G16H 15/00; G16H 30/40

(52) U.S. Cl. CPC G16H 50/20; G06T 7/0012; G16H 15/00; G16H 30/40; G06T 2207/20081; G06T 2207/20084

(57) ABSTRACT

Provided is an image diagnostic system that diagnoses a medical image using an artificial intelligence function. The image diagnostic system includes: a control unit; a diagnosis unit that estimates a diagnosis result using a machine learning model on the basis of an input image; a diagnosis result report output unit that outputs a diagnosis result report on the basis of the diagnosis result; a selection unit that selects a part of a diagnostic content included in the diagnostic result report; and an extraction unit that extracts determination basis information that has affected estimation of the diagnosis content selected, in which the control unit outputs the determination basis information. The image diagnostic system further includes a correction unit that corrects the determination basis information, in which the determination basis information corrected is used for relearning of the machine learning model.

### TECHNICAL FIELD

The technology (hereinafter, “the present disclosure”) disclosed in the present specification relates to an image diagnostic system and an image diagnostic method for diagnosing a medical image such as pathological image data.

### BACKGROUND ART

In order to treat a patient suffering from a disease, it is necessary to identify the pathology. Here, the pathology is a reason, process, and basis for becoming ill. In addition, a doctor who performs pathological diagnosis is referred to as a pathologist. In general, pathological diagnosis is performed by, for example, thinly slicing a lesion collected from a body, performing a treatment such as staining, and diagnosing the presence or absence of a lesion and the type of lesion while observing the lesion with a microscope. Hereinafter, in the present specification, the term “pathological diagnosis” refers to this diagnosis method unless otherwise specified. In addition, an image obtained by observing a thinly sliced lesion with a microscope will be referred to as a “pathological image”, and a digitized pathological image will be referred to as “pathological image data”.

The number of tests using pathological diagnosis tends to increase, but the shortage of pathologists in charge of diagnosis is a problem. The pathologist shortage causes an increase in the workload of the pathologists and an increase in the burden on the patients due to an increase in the period until the diagnosis result is obtained. Therefore, digitization of pathological images, pathological diagnosis using an image analysis function by artificial intelligence, remote diagnosis by pathology online, and the like have been studied.

For example, there has been proposed an information processing device that infers a diagnosis name derived from a medical image on the basis of an image feature amount that is a value indicating a feature of the medical image, infers an image finding expressing the feature of the medical image on the basis of the image feature amount, and presents to a user an image finding and a diagnosis name inferred with an influence of an image feature amount common to the image feature amount influencing the inference of the diagnosis name (see Patent Document 1).

### CITATION LIST

### Patent Document

Patent Document 1: Japanese Patent Application Laid-Open No. 2019-97805
Patent Document 2: Japanese Patent Application Laid-Open No. 2020-38600

### Non Patent Document

Non Patent Document 1: Residuals and Influence in Regression, Cook, R. D. and Weisberg, S <https://conservancy.umn.edu/handle/11299/37076>
Non Patent Document 2: What Uncertainties Do We Need in Bayesian Deep Learning for Computer Vison, NIPS 2017, Alex Kendall and Yarin Gal <https://papers.nips.cc/paper/7141-what-uncertainties-do-we-need-in-bayesian-deep-learning-for-computer-vision.pdf>
Non Patent Document 3: Generative Adversarial Networks, Ian J. Goodfellow et al. <https://arxiv.org/abs/1406.2661>
Non Patent Document 4: Grad-CAM: Visual Explanations from Deep Networks via Gradient-based Localization <https://arxiv.org/abs/1610.02391>
Non Patent Document 5: “Why Should I Trust You?”: Explaining the Predictions of Any Classifier <https://arxiv.org/abs/1602.04938>
Non Patent Document 6: Interpretability Beyond Feature Attribution: Quantitative Testing with Concept Activation Vectors (TCAV)<https://arxiv.org/pdf/1711.11279.pdf>
Non Patent Document 7: Jesse Mu and Jacob Andreas, “Compositional Explanations of Neurons”<34th Conference on Neural Information Processing Systems (NeurIPS 2020), Vancouver, Canada>

### SUMMARY OF THE INVENTION

### Problems to be Solved by the Invention

An object of the present disclosure is to provide an image diagnostic system and an image diagnostic method for diagnosing a medical image using an artificial intelligence function.

### Solutions to Problems

The present disclosure has been made in view of the above problems, and a first aspect thereof is an image diagnostic system including:
a control unit;
a diagnosis unit that estimates a diagnosis result using a machine learning model on the basis of an input image;
a diagnosis result report output unit that outputs a diagnosis result report on the basis of the diagnosis result;
a selection unit that selects a part of a diagnostic content included in the diagnostic result report; and
an extraction unit that extracts determination basis information that has affected estimation of the diagnosis content selected,
in which the control unit outputs the determination basis information.

However, the term, “system”, as used herein refers to a logical assembly of multiple devices (or functional modules that implement specific functions), and each of the devices or functional modules may be or may be not in a single housing.

The image diagnostic system according to the first aspect further includes a correction unit that corrects the determination basis information. Then, the image diagnostic system according to the first aspect may use the corrected determination basis information for relearning the machine learning model.

Furthermore, the information processing device according to the first aspect further includes: an observation data extraction unit that extracts observation data from a diagnosis report for medical image data on the basis of the medical image data, and patient information and an examination value corresponding to the medical image data; and a finding data extraction unit that extracts finding data from the diagnosis report for the medical image data.

In the information processing device according to the first aspect, the diagnosis unit may include an observation data inference unit that infers observation data related to a feature of the input image, and a finding data inference unit that infers finding data related to diagnosis of the input image. In such a configuration, the control unit may calculate a basis of inference of observation data by the observation data inference unit and a basis of inference of finding data by the finding data inference unit. Then, the diagnosis result report output unit may create a diagnosis report of the input image on the basis of the observation data and the finding data inferred by the observation data inference unit and the finding data inference unit, respectively, and the basis calculated by the observation data basis calculation unit and the finding data basis calculation unit.

The information processing device according to the first aspect may further include: an observation data learning unit that learns the first machine learning model using an input image as an explanatory variable and observation data extracted from patient information and an examination value corresponding to the input image as an objective variable; and a finding data learning unit that learns the second machine learning model using an input image as an explanatory variable and finding data extracted from a diagnosis report for the input image as an objective variable.

In addition, a second aspect of the present disclosure is an image diagnostic system that processes information regarding an input image, the image diagnostic system including:
an observation data extraction unit that extracts observation data on the basis of an input image, and patient information and an examination value corresponding to the input image;
a learning unit that performs learning of a machine learning model with an input image and observation data related to the input image as explanatory variables and a diagnosis report of the input image as an objective variable; and
an inference unit that infers a diagnosis report from an input image, and patient information and an examination value corresponding to the input image, using the machine learning model learned.

In addition, a third aspect of the present disclosure is an image diagnostic method for diagnosing an input image, the image diagnostic method including:
an observation data inference step of inferring observation data related to a feature of the input image;
a finding data inference step of inferring finding data related to diagnosis of the input image;
an observation data basis calculation step of calculating a basis of inference of the observation data in the observation data inference step;
a finding data basis calculation step of calculating a basis of inference of the finding data in the finding data inference step; and
a report creation step of creating a diagnosis report of the input image on the basis of the observation data and the finding data inferred in each of the observation data inference step and the finding data inference step and the basis calculated in each of the observation data basis calculation step and the finding data basis calculation step.

### Effects of the Invention

According to the present disclosure, it is possible to provide an information processing device, an information processing method, a computer program, and a medical diagnosis system that perform processing for supporting creation of a diagnosis report of a pathological image using an artificial intelligence function.

Note that the effects described in the present specification are merely examples, and the effects brought by the present disclosure are not limited thereto. Furthermore, the present disclosure may further provide additional effects in addition to the effects described above.

Other objects, features, and advantages of the present disclosure will become apparent from the detailed description based on the embodiments described later and the accompanying drawings.

### BRIEF DESCRIPTION OF DRAWINGS

FIG. 1 is a diagram illustrating a functional configuration example of a medical diagnosis system 100.

FIG. 2 is a diagram illustrating a mechanism for accumulating learning data for model learning.

FIG. 3 is a diagram illustrating an operation of the medical diagnosis system 100 including data adjustment processing by a data adjustment device 200.

FIG. 4 is a diagram illustrating a configuration example of an additional data generation unit 313 using a GAN.

FIG. 5 is a diagram illustrating a conceptual diagram of an observation data inference unit 111 and a finding data inference unit 112.

FIG. 6 is a diagram illustrating a basis calculation result of the observation data inference unit 111.

FIG. 7 is a diagram illustrating a basis calculation result of the observation data inference unit 111.

FIG. 8 is a diagram illustrating a basis calculation result of the finding data inference unit 112.

FIG. 9 is a diagram illustrating a neural network model learned to estimate an output error.

FIG. 10 is a diagram illustrating a configuration example of a diagnosis report 1000.

FIG. 11 is a diagram illustrating a configuration example (a screen indicating a basis of observation data) of a diagnosis report 1000.

FIG. 12 is a diagram illustrating a configuration example (a screen indicating a basis of observation data) of a diagnosis report 1000.

FIG. 13 is a diagram illustrating a configuration example (a screen indicating a basis of finding data) of a diagnosis report 1000.

FIG. 14 is a diagram illustrating a configuration example of a diagnosis report 1400 including information on reliability of observation data and finding data.

FIG. 15 is a diagram illustrating a configuration example of a diagnosis report in which determination bases of all observation data and finding data are simultaneously displayed.

FIG. 16 is a diagram illustrating an editing operation example of a diagnosis report (an example of correcting a basis of feature data).

FIG. 17 is a diagram illustrating an editing operation example of a diagnosis report (an example of correcting a basis of feature data).

FIG. 18 is a diagram illustrating an editing operation example of a diagnosis report (an example of deleting finding data).

FIG. 19 is a diagram illustrating an editing operation example of a diagnosis report (an example of deleting finding data).

FIG. 20 is a diagram illustrating an editing operation example of a diagnosis report (an example of adding feature data and a basis thereof to a diagnosis report).

FIG. 21 is a diagram illustrating an editing operation example of a diagnosis report (an example of adding feature data and a basis thereof to a diagnosis report).

FIG. 22 is a diagram illustrating an editing operation example of a diagnosis report (an example of inputting a missing value).

FIG. 23 is a diagram illustrating an editing operation example of a diagnosis report (an example of inputting a missing value).

FIG. 24 is a diagram illustrating an editing operation example of a diagnosis report (an example of giving a name to a feature amount on pathological image data).

FIG. 25 is a diagram illustrating an editing operation example of a diagnosis report (an example of giving a name to a feature amount on pathological image data).

FIG. 26 is a flowchart illustrating a processing operation in a learning phase of the medical diagnosis system 100.

FIG. 27 is a flowchart illustrating a processing operation in an inference phase of the medical diagnosis system 100.

FIG. 28 is a diagram illustrating a functional configuration example of a medical diagnosis system 2800.

FIG. 29 is a diagram illustrating a functional configuration example of a medical diagnosis system 2900.

FIG. 30 is a diagram illustrating a state in which a pathologist performs pathological diagnosis.

FIG. 31 is a diagram illustrating a configuration example of an information processing device 3100.

FIG. 32 is a diagram schematically illustrating an overall configuration of a microscope system.

FIG. 33 is a diagram illustrating an example of an imaging method.

FIG. 34 is a diagram illustrating an example of an imaging method.

FIG. 35 is a flowchart illustrating a processing procedure for inferring (diagnosing) observation data while prompting a user to input a missing value.

### MODE FOR CARRYING OUT THE INVENTION

Hereinafter, the present disclosure will be described in the following order with reference to the drawings.
A. Overview
B. System Configuration
C. Learning Data
D. Configuration of Machine Learning Model
E. Basis Calculation
F. Reliability Calculation
G. Creation and Presentation of Diagnosis Report
H. Adoption, Modification, and Editing of Diagnosis Report
I. Operation in Learning Phase
J. Operation in Inference Phase
K. Modifications
L. Configuration Example of Information Processing Device
M. Microscope System

### A. Overview

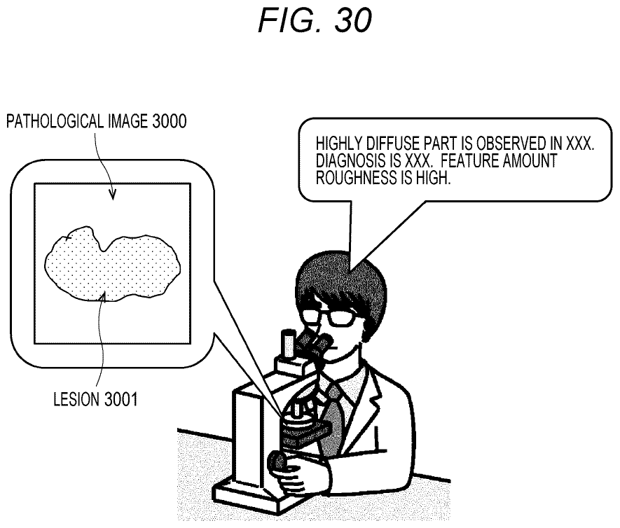

The pathological diagnosis is, for example, a method in which a lesion collected from a body is thinly sliced and subjected to a treatment such as staining, and the presence or absence of a lesion and the type of lesion are diagnosed while being observed using a microscope. FIG. 30 illustrates a state in which a pathologist performs pathological diagnosis. In the example illustrated in FIG. 30, the pathologist is observing a pathological image indicated by reference numeral 3000 using a microscope.

The pathological image 3000 includes a stained lesion 3001. For the pathological image 3000, for example, the pathologist creates a diagnosis report “A highly diffuse part is observed in XXX. The diagnosis is YYY. The feature amount roughness is high.” In general, the diagnosis report includes finding data including a pathological diagnosis result or the like and observation data regarding a tissue of a pathological image. In the case of this example, “Diagnosis name: YYY.” corresponds to finding data, and pathological feature amounts such as “diffuseness: high” and “feature amount: roughness” correspond to observation data. Then, the diagnosis report is recorded in the electronic medical record in association with the pathological image data together with the patient information (age, sex, smoking history, and the like) and the examination value (blood test data, tumor marker, and the like).

If the diagnosis report for the pathological image data can be automatically created using the artificial intelligence function, the workload of the pathologist is reduced. In addition, instead of adopting the diagnosis report automatically created by the artificial intelligence as it is, the pathologist determines the final adoption and corrects or adds to the diagnosis report as necessary, thereby supporting the pathological diagnosis and leading to a reduction in the workload of the pathologist.

Therefore, the present disclosure proposes a medical diagnosis system that supports creation of a diagnosis report of pathological image data using artificial intelligence. In the medical diagnosis system according to the present disclosure, first, a machine learning model is caused to learn a diagnosis report, patient information, examination value data, and pathological image data.

Specifically, diagnosis data related to a diagnosis result and observation data related to a pathological feature amount are extracted from the diagnosis report, and learning of a machine learning model is performed using the diagnosis data and the pathological image data, the observation data and the pathological image data, the patient information, and the examination value as explanatory variables and the finding data and the observation data as objective variables. Then, at the time of pathological diagnosis, the pathological image data to be diagnosed is input to the learned machine learning model, the finding data and the observation data are inferred, and the finding data and the observation data are combined to create a diagnosis report.

In addition, in the case of performing diagnosis of a pathological image by artificial intelligence, it is difficult for a pathologist to determine whether to adopt a diagnosis report if the artificial intelligence is black-boxed and the basis of the determination is not clear. In addition, simply explaining the basis of the diagnosis by the artificial intelligence using eXplainable AI (XAI) technology is not always enough to convince the doctor. On the other hand, in the medical diagnosis system according to the present disclosure, a result of inferring observation data related to a feature of a pathological image together with finding data related to diagnosis using a learned machine learning model is presented together with a basis of inference of each of the finding data and the observation data. Therefore, the pathologist can appropriately evaluate the diagnosis report by the artificial intelligence on the basis of the presented finding data related to diagnosis and observation data related to the features of the pathological image and each basis, and can perform the final diagnosis with high accuracy (or with confidence).

In addition, since medical data includes a wide variety of data such as test items and medical interview, even if a machine learning model is learned by supervised learning, all data necessary for diagnosis are not necessarily prepared, and there is a possibility that a missing value occurs when diagnosis (inference of observation data) is performed using the learned machine learning model. On the other hand, in the medical diagnosis system according to the present disclosure, when the basis for inferring the observation data from the pathological image data is calculated using the learned machine learning model, it is possible to detect that the important basis data is missing and prompt the user (pathologist or the like) to input the missing important variable. For example, in a case where a diagnosis of lung cancer is made, if an important variable is the number of years of smoking by machine learning and if the number of years of smoking is not entered as patient data, it is prompted to complete the entry. Therefore, according to the medical diagnosis system according to the present disclosure, by performing inference again using the missing value input by the user, it is possible to infer observation data by arranging all necessary data.

As a method of calculating important data by machine learning, for example, in the case of a classical machine learning method, there is a method of calculating the importance of each variable (observation data) by using the RandomForest method or LIME which is one of DNN techniques. The important basis data here is, for example, the importance of the above variables calculated and delimited by a certain threshold, and if a variable of high importance is missing as data, the user is prompted to enter it. When the missing value is input, the missing value is filled and then inference is performed again.

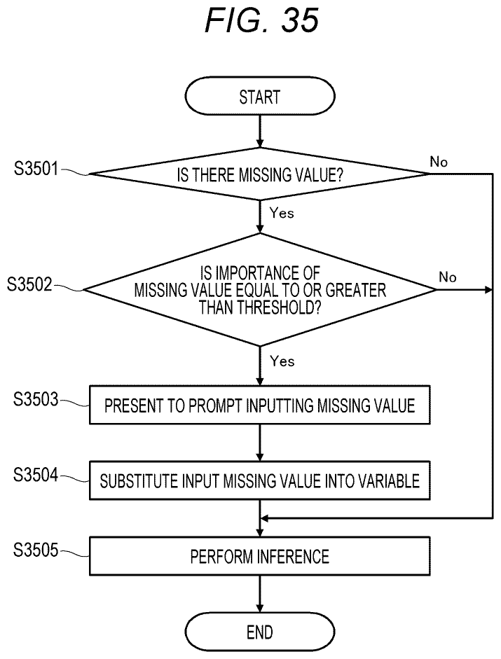

FIG. 35 illustrates a processing procedure for inferring (diagnosing) observation data while prompting the user to input a missing value in the form of a flowchart. First, it is checked whether there is a missing value in the input (step S3501), and in a case where there is a missing value (Yes in step S3501), the importance of the missing value is calculated, and it is further checked whether the importance is equal to or greater than the threshold (step S3502). Then, in a case where the importance of the missing value is equal to or greater than the threshold (Yes in step S3502), the missing value is presented to the user to prompt the user to input the missing value (step S3503), and the missing value input by the user is substituted into the variable (step S3504). In this way, there is no missing value with high importance, and inference (diagnosis) of observation data is performed (step S3505). The calculation timing for calculating the importance of the variable may be performed each time immediately after the learning data is added, or may be periodically performed after a certain amount is accumulated.

### B. System Configuration

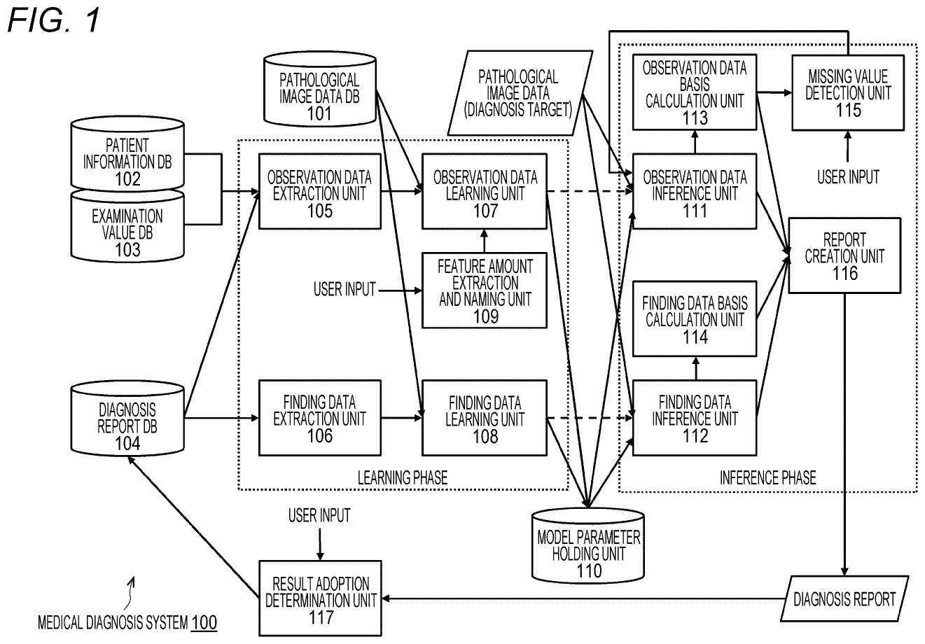

FIG. 1 schematically illustrates a functional configuration example of a medical diagnosis system 100 to which the present disclosure is applied. The medical diagnosis system 100 is configured to mainly perform inference of medical image data such as a pathological image or create or support creation of a diagnosis report using an artificial intelligence function. Specifically, the artificial intelligence function is configured by a machine learning model such as a convolutional neural network (CNN). The operation of the medical diagnosis system 100 is roughly divided into a learning phase and an inference phase. In the learning phase, the machine learning model is learned using medical image data such as a pathological image, patient information, an examination value, and the like as explanatory variables, and using a diagnosis report as an objective variable. In addition, in the inference phase, the diagnosis report is inferred from the medical image data such as the pathological image using the learned machine learning model acquired through the learning phase. In the present embodiment, it is assumed that deep learning is performed using an enormous amount of learning data and inference is performed using a deep neural network (DNN) in the inference phase.

The medical diagnosis system 100 uses two machine learning models of a machine learning model for inferring the finding data from the pathological image data and a machine learning model for inferring the observation data from the pathological image data. Therefore, in the learning phase, learning of these two machine learning models is performed.

In the learning of these two machine learning models, pathological image data, patient information, an examination value, and a diagnosis report are used as explanatory variables. The medical diagnosis system 100 includes a pathological image data DB 101 obtained by converting each of these data into a database (DB), a patient information DB 102, an examination value DB 103, and a diagnosis report DB 104. It is assumed that the pathological image data, the patient information and the examination value of the corresponding patient, and the diagnosis report for the pathological image data are associated with each other.

The patient information includes information associated with the corresponding patient, such as age, sex, height, weight, and medical history. The examination value is data of a blood test, a value of a tumor marker (CA 19-1, CEA, and the like) in blood, or the like, and includes an observation value that can be quantified from blood, tissue, or the like collected from the corresponding patient. The diagnosis report is a report created by a pathologist performing pathological diagnosis on pathological image data, and basically is constituted by natural language, that is, character (text) data.

It is assumed that pathologists all over the country or all over the world perform pathological diagnosis of pathological image data using, for example, the medical system disclosed in Patent Document 2. Then, the pathological image data and the diagnosis report for the pathological image data are collected together with the patient information and the examination value, and are accumulated in each database of the pathological image data DB 101, the patient information DB 102, the examination value DB 103, and the diagnosis report DB 104.

An observation data extraction unit 105 reads the diagnosis report, and the patient information and the examination value corresponding to the diagnosis report from each database of the patient information DB 102, the examination value DB 103, and the diagnosis report DB 104, and extracts the observation data representing the pathological feature amount from the diagnosis report (text data). For example, the observation data extraction unit 105 extracts observation data representing a pathological feature amount from the diagnosis report on the basis of the age, sex, height, and weight of the patient included in the corresponding patient information, and the corresponding examination value. In addition, the observation data extraction unit 105 performs natural language processing on the diagnosis report constituted by the text data, and acquires an observable feature and a degree thereof (words expressing a feature of an image of each part of the pathological image data, or the like). In the above-described example illustrated in FIG. 30, the observation data extraction unit 105 extracts a feature of “diffuseness” and a degree of “high”, and a feature of “roughness” and a degree of “high”. The observation data extraction unit 105 may perform observation data extraction processing using a machine learning model learned to detect a word or a phrase representing a feature of an image from text data.

A finding data extraction unit 106 performs natural language processing on the diagnosis report constituted by characters to extract data related to diagnosis or finding. The data related to diagnosis is, for example, a diagnosis result of what diagnosis the case of the pathological image data has made. In the example illustrated in FIG. 30 described above, the word of the disease name “YYY” is extracted by the finding data extraction unit 106. The finding data extraction unit 106 may perform the extraction processing of the finding data using a machine learning model learned to infer the disease name and the symptom from the pathological image data.

An observation data learning unit 107 performs learning processing of the first machine learning model with the pathological image data as an explanatory variable and the observation data as an objective variable. Specifically, the observation data learning unit 107 performs learning processing of the first machine learning model so as to infer the observation data from the pathological image data using, as the learning data, a data set in which the pathological image data read from the pathological image data DB 101 is the input data and the observation data extracted from the diagnosis report on the basis of the corresponding patient information and examination value read from each of the patient information DB 102 and the examination value DB 103 by the observation data extraction unit 105 is the correct answer label. The first machine learning model includes, for example, a neural network having a structure that mimics human neurons. The observation data learning unit 107 calculates a loss function based on an error between a label output from the first machine learning model under learning with respect to input data and a correct answer label, and performs learning processing of the first machine learning model so as to minimize the loss function.

A finding data learning unit 108 performs learning processing of the second machine learning model with the pathological image data as an explanatory variable and the finding data as an objective variable. The finding data learning unit 108 performs learning processing of the second machine learning model so as to infer the finding data from the pathological image data using, as the learning data, a data set in which the pathological image data read from the pathological image data DB 101 is input data and the finding data extracted by the finding data extraction unit 106 from the corresponding diagnosis report read from the diagnosis report DB 104 is a correct answer label. The second machine learning model includes, for example, a neural network having a structure that mimics human neurons. The finding data learning unit 108 calculates a loss function based on an error between a label output from the second machine learning model under learning with respect to input data and a correct answer label, and performs learning processing of the second machine learning model so as to minimize the loss function. Note that, for learning of the finding data, learning may be performed so that the finding data can be output with not only the finding data but also the observation data as an input using the technology of DataCaptioning.

The observation data learning unit 107 and the finding data learning unit 108 perform learning processing of the first machine learning model and the second machine learning model by updating the model parameters so as to output the correct answer label to the captured pathological image data. The model parameter is a variable element that defines the behavior of the machine learning model, and is, for example, a weighting coefficient or the like given to each neuron of the neural network. In the back propagation method, a loss function is defined on the basis of an error between a value of an output layer of a neural network and a correct diagnosis result (correct answer label), and model parameters are updated so that the loss function is minimized using a steepest descent method or the like. Then, the observation data learning unit 107 and the finding data learning unit 108 store the model parameters of each machine learning model obtained as the learning result in a model parameter holding unit 110.

Specifically, a CNN is used as a machine learning model that performs observation data inference and finding data inference, respectively, and the machine learning model includes a feature amount extraction unit that extracts a feature amount of an input image and an image classification unit that infers an output label (in the present embodiment, observation data and finding data) corresponding to the input image on the basis of the extracted feature amount. The former feature amount extraction unit includes a “convolution layer” that extracts an edge or a feature by performing convolution of an input image by a method of limiting connection between neurons and sharing a weight, and a “pooling layer” that deletes information on a position that is not important for image classification and gives robustness to the feature extracted by the convolution layer. Furthermore, at the time of learning, it is possible to perform “transfer learning” in which one of the observation data learning unit 107 and the finding data learning unit 108 fixes the result of learning of the feature amount extraction unit in the preceding stage, and the other causes only the image classification unit in the subsequent stage to learn another problem.

The observation data learning unit 107 may perform learning on the basis of not only the diagnosis report and the observation value (patient information and examination value) extracted by the observation data extraction unit 105 but also the composite variable synthesized by the user (pathologist or the like). The composite variable referred to herein may be, for example, a feature amount extracted by the CNN from the input image in the middle of learning, or may be a variable obtained by simply combining a plurality of observation values such as age and sex. Furthermore, the user may define the composite variable. For example, the pathologist may designate a region with respect to the pathological image data during pathological diagnosis, and may input how the feature amount is, such as “bumpy feeling is high”.

A feature amount extraction and naming unit 109 newly extracts a variable on the basis of the synthesis of the observation values or the definition of the user, and assigns the variable to the name of the corresponding image feature amount. For example, the feature amount extraction and naming unit 109 extracts a variable, presents the variable to the user, gives a name to the feature such as “bumpy feeling” given by the user, and outputs the feature to the observation data learning unit 107. In this case, the observation data learning unit 107 performs learning using the name input from the feature amount extraction and naming unit 109 as the objective variable. Note that the feature amount extraction and naming unit 109 may extract a portion that is pathologically characteristic in the input pathological image data and generate a response sentence describing the portion by using an attention model (see, for example, Non Patent Document 7) that learns an attention portion instead of the input from the user.

In the inference phase, an observation data inference unit 111 reads the model parameters of the first machine learning model learned by the observation data learning unit 107 from the model parameter holding unit 110, and makes the first machine learning model with the pathological image data as an explanatory variable and the observation data as an objective variable available. Similarly, a finding data inference unit 112 reads the model parameters of the second machine learning model learned by the finding data learning unit 108 from the model parameter holding unit 110, and makes the second machine learning model with the pathological image data as an explanatory variable and the finding data as an objective variable available. Note that, instead of temporarily storing the model parameters in the model parameter holding unit 110, the model parameters obtained by the learning processing of the observation data learning unit 107 may be directly set in the observation data inference unit 111, and the model parameters obtained by the learning processing of the finding data learning unit 108 may be directly set in the finding data inference unit 112.

Then, the pathological image data of the diagnosis target captured from the image capturing unit (not illustrated) is input to each of the observation data inference unit 111 and the finding data inference unit 112. The observation data inference unit 111 infers the input pathological image data and outputs a label of the corresponding observation data. Similarly, the finding data inference unit 112 infers the input pathological image data and outputs a label of the corresponding finding data.

In addition, the observation data inference unit 111 and the finding data inference unit 112 may output the estimated reliability of the output label. Details of a method of calculating the reliability of the output label in the neural network model will be described later.

An observation data basis calculation unit 113 calculates the basis (that is, the basis that the machine learning model has determined the output label) of the observation data estimated by the observation data inference unit 111 using the learned first machine learning model. In addition, a finding data basis calculation unit 114 calculates the basis (that is, the basis that the machine learning model has determined the output label) of the finding data estimated by the finding data inference unit 112 using the learned second machine learning model.

Each of the observation data basis calculation unit 113 and the finding data basis calculation unit 114 can calculate an image in which the determination basis of each of the diagnosis and the differential diagnosis using the learned machine learning model is visualized in the observation data inference unit 111 and the finding data inference unit 112 using an algorithm such as Gradient-weighted Class Activation Mapping (Grad-CAM) (see, for example, Non Patent Document 4), LOCAL Interpretable model-agnostic Explanations (LIME) (see, for example, Non Patent Document 5), Shapley Additive exPlanations (SHAP) which is an evolution form of LIME, Testing with Concept Activation Vectors (TCAV) (see, for example, Non Patent Document 6), and an attention model. The observation data basis calculation unit 113 and the finding data basis calculation unit 114 may calculate the basis of inference using the same algorithm, or may calculate the basis of inference using different algorithms. However, details of the basis calculation method using the Grad-Cam, LIME/SHAP, TCAV, and attention models will be described later.

Note that the finding data basis calculation unit 114 may target a phrase of observation data related to a feature of a pathological image (symptom or the like) as a basis of finding data related to diagnosis. In such a case, a knowledge database (rule) for associating a symptom with a diagnosis is prepared in advance, and when calculating the basis of finding data, the finding data basis calculation unit 114 is only required to query the knowledge database about the symptom associated with the finding data, find observation data (for example, phrases such as “diffuseness in XXX”) corresponding to the hit symptom from the output label of the observation data inference unit 111, and acquire the observation data as the basis of the finding data.

In addition, the observation data basis calculation unit 113 and the finding data basis calculation unit 114 may target the examination value of the patient (blood test data, tumor marker, and the like) as the basis of the observation data or the finding data. In such a case, a knowledge database (rule) for associating the examination value with the observation data and the finding data is prepared in advance, and the observation data basis calculation unit 113 and the finding data basis calculation unit 114 are only required to query the knowledge database for the examination value associated with the observation data or the finding data and acquire the hit examination value as the basis of the observation data or the finding data when calculating the basis of the observation data or the finding data. For example, in a case where the observation data inference unit 111 infers that the diffuseness of the site XXX in the pathological image data is high, the observation data basis calculation unit 113 can find a basis that “the tumor marker RRR is high” for “there is a highly diffuse part in XXX” by referring to the knowledge database.

When the observation data basis calculation unit 113 calculates the basis of the observation data inference by the observation data inference unit 111, a missing value detection unit 115 detects that the importance of the variable (for example, age, a value of a tumor marker, and the like) serving as the basis is missing, and prompts the user (such as a pathologist) to input the missing important variable. As a method of calculating important data by machine learning, for example, in the case of a classical machine learning method, there is a method of calculating the importance of each variable (observation data) by using the RandomForest method or LIME which is one of DNN techniques. Then, when the user inputs the missing value, the missing value detection unit 115 inputs the input missing value to the observation data inference unit 111, and the observation data inference unit 111 infers the observation data again from the pathological image data. For example, when the missing value detection unit 115 detects that the importance of the variable that is the basis of the inference of the observation data such as the age and the value of the tumor marker is missing, it prompts the user to input the missing important variable. In addition, in a case where a diagnosis of lung cancer is made, assuming that an important variable is the number of years of smoking by machine learning, if the number of years of smoking is not entered as patient data, the user is prompted to complete the entry. Then, the missing value detection unit 115 inputs the missing value input by the user to the observation data inference unit 111, and the observation data inference unit 111 performs inference again on the same pathological image data. Note that the calculation of the importance of the variable is adopted by a result adoption determination unit 117 (described later) and is calculated using the learning data held in the diagnosis report DB 104.

The observation data and the finding data inferred by the observation data inference unit 111 and the finding data inference unit 112 from the pathological image data to be diagnosed are input to a report creation unit 116. In addition, each basis of the observation data and the finding data calculated by the observation data basis calculation unit 113 and the finding data basis calculation unit 114 is also input to the report creation unit 116.

The report creation unit 116 creates a diagnosis report of the pathological image data on the basis of the observation data and the finding data input from the observation data inference unit 111 and the finding data inference unit 112 and the respective bases calculated by the observation data basis calculation unit 113 and the finding data basis calculation unit 114. The report creation unit 116 may create a diagnosis report constituted by natural language (fluent sentence) from observation data and finding data constituted by fragmentary words and phrases using a language model such as Generative Pre-Training 3 (GPT-3), for example.

The created diagnosis report is displayed, for example, on a screen of a monitor display (not illustrated). A user (such as a pathologist) can check the contents of the diagnosis report via the display screen of the monitor display. In addition, the user (pathologist or the like) can appropriately browse the basis of each of the observation data and the finding data described in the diagnosis report. In addition, in a case where the observation data inference unit 111 and the finding data inference unit 112 output the reliability of the output label, the reliability of each of the observation data and the finding data on the diagnosis report may be presented together. The configuration of the screen displaying the diagnosis report will be described later.

The diagnosis report includes not only the finding data related to diagnosis for the pathological image data and the basis of inference thereof but also the observation data related to the features of the pathological image data and the basis of inference thereof. Therefore, the user (pathologist) can determine adoption of the diagnosis report in a more satisfactory manner.

The user (pathologist) can input the final adoption of the diagnosis report created by the report creation unit 116 to the result adoption determination unit 117. Then, the result adoption determination unit 117 receives an input from the user, stores the adopted diagnosis report in the diagnosis report DB 104, and uses the diagnosis report as learning data in the subsequent learning processing. In addition, the result adoption determination unit 117 discards the diagnosis report not adopted by the user and does not add the diagnosis report to the diagnosis report DB 104.

The result adoption determination unit 117 may receive the user input and provide an editing environment in which the user (pathologist) corrects or edits the diagnosis report as well as adoption of the diagnosis report created by the report creation unit 116. The correction and editing of the diagnosis report includes correction of observation data, correction of finding data, correction of basis of observation data and finding data, addition of observation data, input of added observation data, and the like. In addition, observation data and finding data corrected or added by the user (pathologist) with respect to the diagnosis report automatically created by the report creation unit 116 and the basis thereof can be utilized for learning data of relearning.

The result adoption determination unit 117 may include not only a simple user input instructing adoption of the diagnosis report but also a use interface (UI) or user eXperience (UX) for the user to perform relatively advanced correction or editing work of the diagnosis report. In addition, the UI/UX may prepare a template or the like for supporting or simplifying the editing work of the diagnosis report.

As described above, the medical diagnosis system 100 according to the present disclosure is configured to learn a diagnosis report for pathological image data by dividing the diagnosis report into observation data related to a feature of an image and finding data related to image diagnosis. Therefore, the medical diagnosis system 100 can automatically create the diagnosis report while presenting the observation data and the finding data inferred from the pathological image data to be diagnosed together with each basis.

Note that the learning phase and the inference phase may be realized on individual information processing devices (personal computers or the like). Alternatively, the learning phase and the inference phase may be realized on one information processing device.

### C. Learning Data

The learning data for learning the machine learning model used in the medical diagnosis system 100 includes digitized pathological image data, a diagnosis report by a pathologist for the pathological image data, and a data set obtained by combining patient information and an examination value. For example, the pathology data and the diagnosis report are recorded in the electronic medical record for each patient together with the patient information and the examination value.

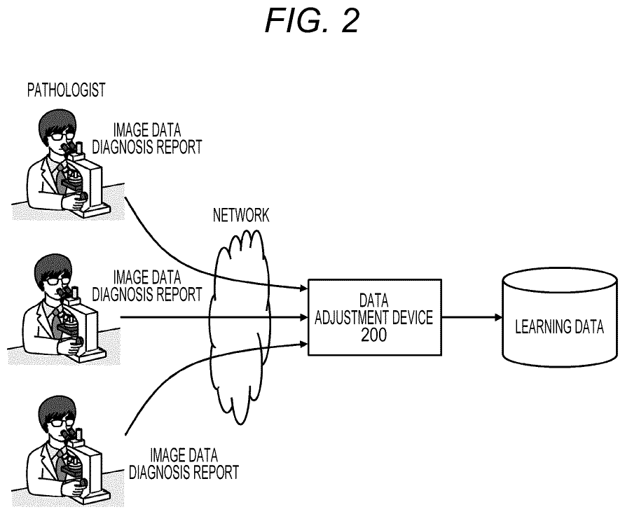

FIG. 2 schematically illustrates a mechanism for collecting pathological image data and diagnosis reports diagnosed by pathologists scattered all over the country or all over the world and accumulating learning data for model learning. Each pathologist may perform pathological diagnosis of pathological image data using, for example, the medical system disclosed in Patent Document 2. Then, the pathological image data pathologically diagnosed by each pathologist and the diagnosis report thereof are collected on a cloud through a wide area network such as the Internet, for example. Note that, as already described with reference to FIG. 1, the processing of extracting observation data from the diagnosis report, the patient information, and the examination value, and the processing of extracting finding data from the diagnosis report are necessary as preprocessing for acquiring learning data, but illustration is omitted in FIG. 2 for simplification of description.

Deep learning of a machine learning model requires a huge amount of learning data. All the data sets collected on the cloud may be utilized for the learning data. However, among the collected data sets, data adjustment processing such as removal of a harmful data set such as a data set having a low degree of contribution to learning of the machine learning model and investigation of uncertainty of the machine learning model may be performed by a data adjustment device 200 to construct learning data for deep learning.

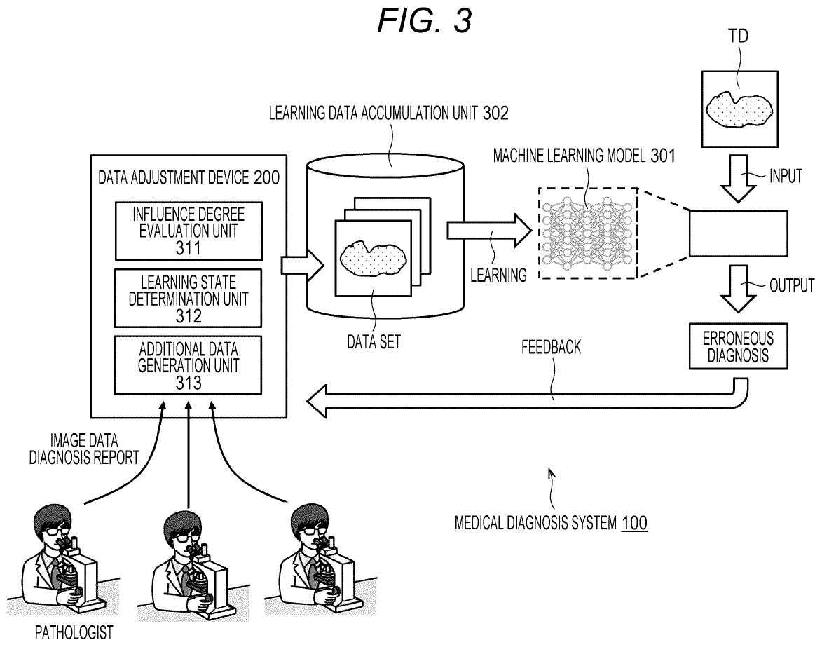

FIG. 3 conceptually illustrates the operation of the medical diagnosis system 100 including the data adjustment processing by the data adjustment device 200.

A learning data accumulation unit 302 accumulates learning data including a data set or the like obtained by combining pathological image data diagnosed by pathologists, observation data extracted from diagnosis reports or the like, and finding data. The observation data learning unit 107 and the finding data learning unit 108 perform learning processing (deep learning) of a machine learning model 301 configured by a neural network (CNN or the like) using the data set.

Test data (TD) such as pathological image data is input to the machine learning model 301 in the learning process, correctness of an output label (inference result of observation data and finding data with respect to the input pathological image data) from the machine learning model 301 is determined, and the information is fed back if the erroneous diagnosis occurs, thereby learning the machine learning model 301.

The data adjustment device 200 includes an influence degree evaluation unit 311, a learning state determination unit 312, and an additional data generation unit 313. The influence degree evaluation unit 311 evaluates the influence degree of each data set collected through a network or the like on the machine learning model 311. A data set having a high degree of influence is useful learning data, but a data set having a low degree of influence is harmful as learning data and may be removed. In addition, the learning state determination unit 312 determines whether the accuracy cannot be further improved due to the state of learning of the machine learning model 301, specifically, the limit of deep learning, or the accuracy is not obtained due to the lack of learning data (whether the accuracy can be further improved by relearning). In addition, the additional data generation unit 313 generates additional learning data from the already acquired learning data (accumulated in the learning data accumulation unit 101) without depending on the collection of a new data set from the pathologist. Hereinafter, processing of each unit will be described in more detail.

### C-1. Influence Degree Evaluation

Here, a method of evaluating the degree of influence of each data set collected through a network or the like on the machine learning model 301, which is performed by the influence degree evaluation unit 311, will be described.

The data set z is data in which an output label (diagnosis result) y is associated with an input (pathological image data) x. As illustrated in the following formula (1), it is assumed that there are n data sets.

[Math. 1]

z₁,z₂, . . . ,zₙ zᵢ=(xᵢ,yᵢ)∈X×Y (1)

When the model parameter of the machine learning model 301 is θ∈Θ, assuming that the loss of the data set z is L (z, θ), the experience loss in all the n data sets can be expressed as the following formula (2).

$$\begin{matrix}[ Math . 2 ] & \\ \frac{1}{n} {\sum }_{i = 1}^{n} L ( {z}_{i} , θ ) & ( 2 )\end{matrix}$$

Learning of the machine learning model 301 means finding model parameters that minimize the experience loss. Therefore, the model parameters obtained as a result of performing the learning of the machine learning model 301 using the n data sets illustrated in the above formula (1) can be expressed as the following formula (3). However, as illustrated on the left side of the formula (3), in a case where “\hat{ }” is added above the parameter “θ”, it is assumed that the parameter “θ” represents a prediction value. Hereinafter, in the text, the prediction value of the parameter θ is expressed as “θ\hat{ }” in which “\hat{ }” is described following “θ”.

$$\begin{matrix}[ Math . 3 ] & \\ \hat{θ} = arg {min}_{θϵΘ} \frac{1}{n} {\sum }_{i = 1}^{n} L ( {z}_{i} , θ ) & ( 3 )\end{matrix}$$

Next, the influence on the learning of the machine learning model 301 in a case where there is no data set z of a certain training point will be considered. The model parameters of the machine learning model 301 when the learning processing is performed by removing the data set z of the training points can be expressed as the following formula (4).

$$\begin{matrix}[ Math . 4 ] & \\ {\hat{θ}}_{- z} = arg {min}_{θϵΘ} \frac{1}{n} {\sum }_{{z}_{i} \neq z} L ( {z}_{i} , θ ) & ( 4 )\end{matrix}$$

The influence degree of the data set z of the training point is a difference between model parameters obtained by performing the learning processing when the data set z is removed and when all n data sets including the data set z are used. This difference is expressed by the following formula (5).

[Math. 5]

\hat{θ}_{-z}−\hat{θ} (5)

If the data set z of a specific data point is removed and the model parameters are relearned, the calculation cost is very high. Therefore, the influence degree evaluation unit 311 effectively approximates and calculates the influence degree z of the data set without recalculating using influence functions (see Non Patent Document 1). Specifically, the change in the parameter is calculated assuming that the input data (image) of the data set z is weighted by the minute value ε. Here, a new parameter “θ_{ε,z}\hat{ }” as illustrated on the left side thereof is defined using the following formula (6).

$$\begin{matrix}[ Math . 6 ] & \\ {\hat{θ}}_{ε , z} ≡ arg {min}_{θ ϵ Θ} \frac{1}{n} {\sum }_{{z}_{i} \neq z} L ( {z}_{i} , θ ) + ε L ( z , \hat{θ} ) & ( 6 )\end{matrix}$$

Then, the influence function corresponding to the data set z can be expressed using the following formulas (7) and (8).

$$\begin{matrix}[ Math . 7 ] & \\ {{I}_{up , params} ( z ) ≡ \frac{d {\hat{θ}}_{ε , z}}{d ε} \vert}_{ε = 0} = - {H}_{\hat{θ}}^{- 1} {∇}_{θ} L ( z , \hat{θ} ) & ( 7 )\end{matrix}$$

$$\begin{matrix}[ Math . 8 ] & \\ {H}_{\hat{θ}} = \frac{1}{n} {\sum }_{i = 1}^{n} {∇}_{θ}^{2} L ( {z}_{i} , θ ) & ( 8 )\end{matrix}$$

The above formula (7) is an influence function corresponding to the data set z, and represents, for example, a change amount of the model parameter θ \hat{ } with respect to the minute weight ε. In addition, the above formula (8) represents Hessian (Hessian matrix). Here, it is assumed that the matrix is a Hessian matrix having a positive definite value, and an inverse matrix also exists. Assuming that removing the data set z at a certain training point is the same as being weighted by “ε=−1/n”, the change in the model parameters when removing the data set z can be approximated by the following formula (9).

$$\begin{matrix}[ Math . 9 ] & \\ {\hat{θ}}_{- z} - \hat{θ} \approx - \frac{1}{n} {I}_{up , params} ( z ) & ( 9 )\end{matrix}$$

Therefore, the influence degree evaluation unit 311 can measure the influence degree of the data set z without relearning.

$$[ Math . 10 ]$$

$$\begin{matrix}{{I}_{up , loss} ( z , {z}_{test} ) ≡ \frac{d L ( {z}_{test} , {\hat{θ}}_{ε , z} )}{d ε} \vert}_{ε = 0} & ( 10 - 1 ) \\ {= {∇}_{θ} {L ( {z}_{test} , θ )}^{T} \frac{d {θ}_{ε , z}}{d ε} \vert}_{ε = 0} & ( 10 - 2 ) \\ = {∇}_{θ} {L ( {z}_{test} , θ )}^{T} {H}_{θ}^{- 1} {∇}_{θ} L ( z , θ ) & ( 10 - 3 )\end{matrix}$$

In this way, the degree of influence of the weighted data set z at a certain test point zₜₑₛₜ can be formulated. Therefore, the influence degree evaluation unit 311 can measure the influence degree of the data set in the machine learning model 301 by this calculation. For example, the influence of a certain data set on the prediction (loss) of the model can be obtained by the above formula (10-3). The right side of the above formula (10-3) includes a gradient with respect to loss of certain data, an inverse matrix of Hessian, a gradient of loss of certain learning data, and the like.

However, the method of evaluating the degree of influence described in this item C-1 is an example, and the influence degree evaluation unit 311 may measure the degree of influence of the data set by another method.

### C-2. Determination of Learning State

Here, a method of determining the state of learning of the machine learning model 301 performed by the learning state determination unit 312 will be described.

Generally, the inference of the DNN model is highly accurate, but there is a limit to the inference. It is very important to grasp the state of learning of the model, that is, whether the accuracy cannot be further improved due to the limit of deep learning, or whether the accuracy is not obtained due to the lack of learning data (whether the accuracy can be further improved by relearning), in order to use deep learning. However, it is difficult to completely eliminate the uncertainty of deep learning.

The uncertainty of deep learning can be divided into two types: aleatoric uncertainty and epistemic uncertainty. The former case of aleatoric uncertainty is caused by noise due to observation, and is not caused by lack of data. For example, a hidden and invisible image (occlusion) corresponds to the aleatoric uncertainty. Since a mouth of a face of a masked person is originally hidden by the mask, it cannot be observed as data. On the other hand, the latter case of epistemic uncertainty is caused by lack of data, and epistemic uncertainty can be improved if sufficient data exists.

The learning state determination unit 312 clarifies the uncertainty of the machine learning model 301 using Bayesian deep learning (see, for example, Non Patent Document 2). The Bayesian deep learning determines uncertainty of an inference result using a dropout (random invalidation of some model parameters) not only at the time of learning but also at the time of inference. Specifically, when data (pathological image data) is input to the machine learning model 301, the data passes through a missing neuron due to dropout, and an output label characterized by a weight of the path is obtained. However, even if the same data is input, the data passes through different paths and is output, so that the output is dispersed. A large variance of the output means that the uncertainty in the inference of the machine learning model 301 is large, and the uncertainty can be improved by performing learning with sufficient learning data.

Therefore, on the basis of the result of the learning state determination unit 312 determining the learning state on the basis of Bayesian deep learning, the learning unit 102 is only required to end the learning of the machine learning model 301 or continue the learning by adding learning data.

### C-3. Generation of Additional Data

Here, a method of generating additional learning data from existing learning data performed by the additional data generation unit 313 will be described. The additional data generation unit 313 generates additional learning data for relearning the machine learning model 301, for example, in response to a result of the learning state determination unit 312 determining the uncertainty of the machine learning model 301. In addition, the additional data generation unit 313 may generate the additional data by being triggered by erroneous determination of the output label when the test data (TD) is input to the machine learning model 301. The additional data generation unit 313 may generate the additional data on the basis of the test data at that time.

In the present embodiment, it is assumed that the additional data generation unit 313 automatically generates additional learning data using a Generative Adversarial Network (GAN) algorithm (for example, see Non Patent Document 3). The GAN is an algorithm that causes two networks to compete with each other to enhance learning of input data.

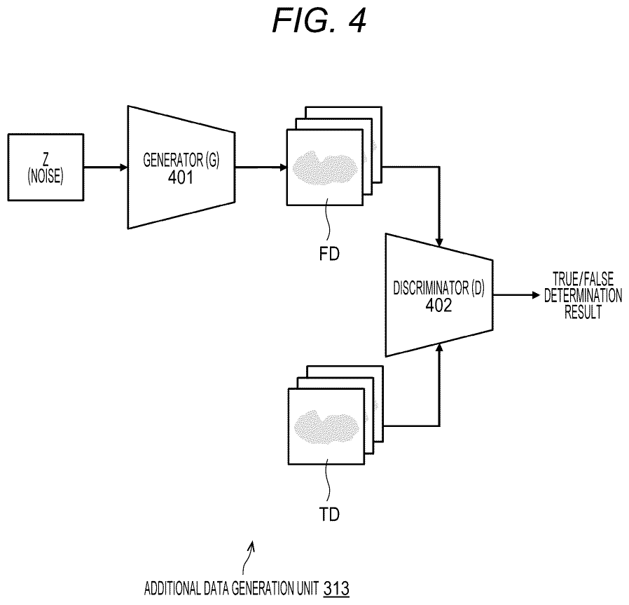

FIG. 4 illustrates a configuration example of the additional data generation unit 313 using the GAN. The additional data generation unit 313 illustrated in FIG. 4 includes a generator (G) 401 and a discriminator (D) 402. The generator 401 and the discriminator 402 are each configured by a neural network model.

The generator 401 adds noise to the pathological image data accumulated in the learning data accumulation unit 101 to generate false pathological image data (Fake Data: FD). On the other hand, the discriminator 402 discriminates between true pathological image data and true/false pathological image data generated by the generator 401. Then, the generator 401 learns to compete with each other to make it difficult for the discriminator 402 to determine the true/false, while the discriminator 402 learns to correctly identify the pathological image data generated by the generator 401, so that they can generate new pathological image data that cannot be authenticated. The process of mutual learning is expressed as the following formula (11).

$$[ Math . 11 ]$$

$${min}_{G} {max}_{D} V ( D , G ) = {𝔼}_{x \sim {p}_{data} ( x )} [ log D ( x ) ] + {𝔼}_{x \sim {p}_{data} ( z )} [ log ( 1 - D ( G ( z ) ) ) ] \qquad ( 11 )$$

In the above formula (11), G corresponds to the generator 401, and D corresponds to the discriminator 402. D determines whether G is authentic or fake and learns to maximize the probability D (x) of correctly labeling. On the other hand, G learns to minimize the probability log (1−D (G (z))) of labeling G as fake in order to cause D to recognize itself as authentic.

In a case where D can be correctly labeled, the value of D (x) increases and the value of log D (x) also increases. Furthermore, D (G (z)) decreases by finding that G is fake. As a result, log (1−D (G (z))) becomes large, and D becomes dominant. On the other hand, in a case where data in which G is close to authentic can be generated, the value of G (z) increases, and the value of D (G (z)) also increases. Furthermore, since D cannot be correctly labeled, the value of D (x) decreases, and the value of log D (x) also decreases. As a result, log (1−D (G (z))) decreases, and G becomes dominant. By repeating such an operation, D and G are alternately updated, and each learning can be deepened.

Of course, the additional data generation unit 313 may generate additional learning data using an algorithm other than the GAN, or may newly collect the pathologist diagnosis result of the pathologist and acquire new learning data.

### D. Configuration of Machine Learning Model

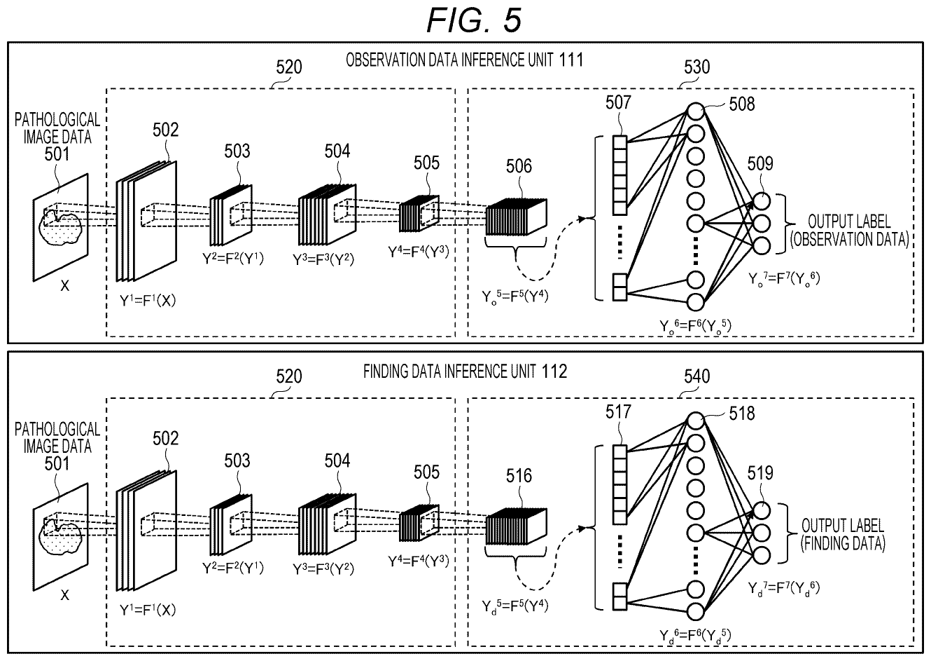

FIG. 5 illustrates a conceptual diagram of the observation data inference unit 111 and the finding data inference unit 112 in the medical diagnosis system 100 illustrated in FIG. 1. As described above, the observation data inference unit 111 and the finding data inference unit 112 use the machine learning models learned by the observation data learning unit 107 and the finding data learning unit 108, respectively. Here, it is assumed that each machine learning model is configured using a multilayer convolutional neural network (CNN), and includes a feature amount extraction unit that extracts a feature amount of an input image and an image classification unit that infers an output label (in the present embodiment, observation data and finding data) corresponding to the input image on the basis of the extracted feature amount. The former feature amount extraction unit includes a “convolution layer” that extracts an edge or a feature by performing convolution of an input image by a method of limiting connection between neurons and sharing a weight, and a “pooling layer” that deletes information on a position that is not important for image classification and gives robustness to the feature extracted by the convolution layer.

In the CNN illustrated in FIG. 5, a range surrounded by a square indicated by reference numeral 520 is a feature amount extraction unit, and performs processing of acquiring an image feature amount of the input pathological image data. Then, each range surrounded by a square indicated by reference numerals 530 and 540 is an image classification unit, and specifies an output label on the basis of the image feature amount (in the present embodiment, the image classification unit 530 infers observation data, and the image classification unit 540 infers finding data).

In FIG. 5, reference numeral 501 denotes an image (pathological image data) that is input data to the CNN. Reference numerals 502, 504, 506 and 516 denote the output of the convolutional layer. Reference numerals 503 and 505 denote the output of the pooling layer. Reference numerals 507 and 517 indicate a state in which the outputs 506 and 516 of the convolution layer are arranged one-dimensionally, respectively, reference numerals 508 and 518 indicate fully connected layers, and reference numerals 509 and 519 indicate output layers that output observation data and finding data that are inference results of class classification, respectively.

During learning, transfer learning can be performed between the observation data learning unit 107 and the finding data learning unit 108. That is, first, in the observation data learning unit 107, the feature amount extraction unit 520 performs learning processing so as to extract the feature amount of the image from the pathological image data, and the image classification unit 930 performs learning processing so as to infer the observation data from the image feature amount. Thereafter, in the finding data learning unit 108, the model parameters learned by the observation data learning unit 107 are fixed for the feature amount extraction unit 520 in the preceding stage, and only the image classification unit 940 in the subsequent stage is caused to learn for another problem of inferring the finding data from the image feature amount.

Note that a stage of the inference process (order of processing of each layer) is denoted by l, an output value in the l-th layer is denoted by Y^{l}, and processing in the l-th layer is denoted by Y^{l}=F^{l} (Y^{l-1}). In addition, Y¹=F¹ (X) is set for the first layer, and Y=F⁷ (Y⁶) is set for the final layer. In addition, a subscript “o” is added to the processing of the image classification unit 530 that classifies the observation data, and a subscript “d” is added to the processing of the image classification unit 504 that classifies the finding data.

### E. Basis Calculation

The observation data basis calculation unit 113 calculates the basis that the observation data inference unit 111 has inferred the observation data, and the finding data basis calculation unit 114 calculates the basis of the finding data inferred by the finding data inference unit 112. Each of the basis calculation units 113 and 114 calculates an image in which the determination basis of each of the inference label and the differential label is visualized using, for example, an algorithm such as Grad-CAM, LIME/SHAP, TCAV, or an attention model.

The Grad-CAM is an algorithm that estimates a place contributing to class classification in the input image data by a method of reversely tracing (calculating contribution of each feature map up to class classification, and perform back propagation using weight thereof) a gradient from a label that is an inference result of class classification in the output layer, and can visualize the place contributing to class classification like a heat map. Alternatively, by holding the positional information of the pixels of the input image data up to the final convolution layer and obtaining the degree of influence of the positional information on the final determination output, a part having a strong influence in the original input image may be displayed by heat map display.

A method of calculating a determination basis on the basis of the Grad-Cam algorithm (a method of generating a heat map) in a case where image recognition is performed on an input image and class c is output in a neural network model such as CNN will be described.

Assuming that the gradient y_{c} of the class c is the activation Aₖ of the feature map, a weight of the importance of the neuron is given as illustrated in the following formula (12).

$$\begin{matrix}[ Math . 12 ] & \\ {α}_{k}^{c} = \frac{1}{Z} {\sum }_{i} {\sum }_{j} \frac{\partial {y}^{c}}{\partial {A}_{ij}^{k}} & ( 12 )\end{matrix}$$

The forward propagation output of the final convolution layer is multiplied by the weight for each channel, and Grad-Cam is calculated via the activation function ReLU as illustrated in the following formula (13).

[Math. 13]

L_{Grad-CAM}^{c}=ReLU(Σₖαₖ^{c}A^{k}) (13)

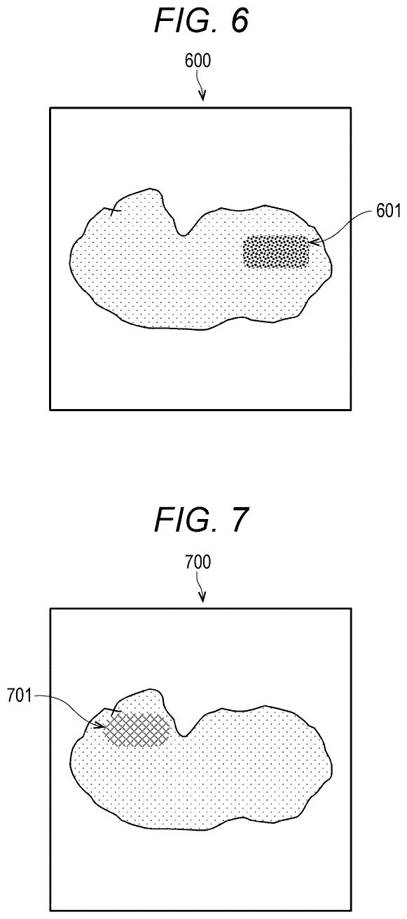

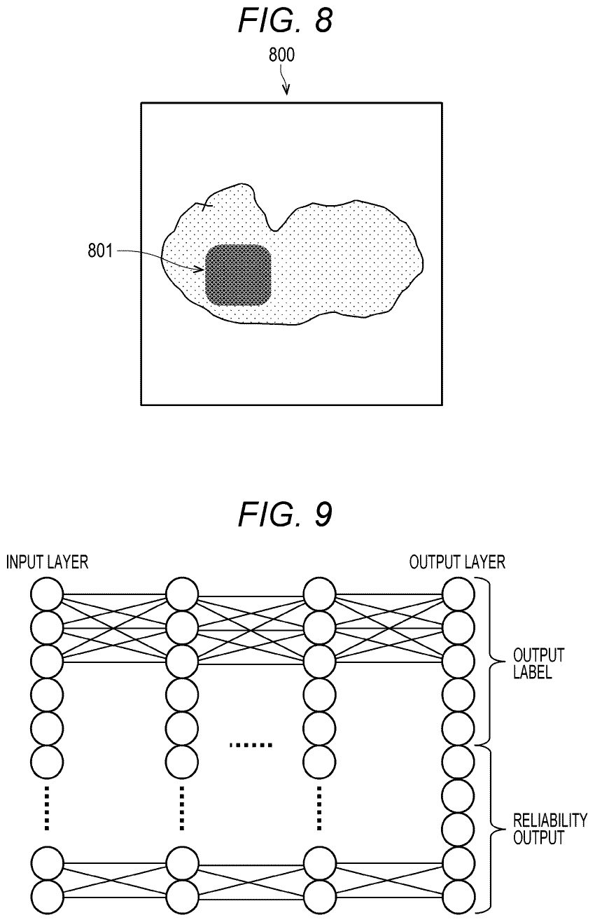

FIG. 6 illustrates an image 600 in which a heat map 601 indicating a portion (that is, highly diffuse part XXX) serving as a basis of inference calculated by the observation data basis calculation unit 113 when the observation data inference unit 111 inferred observation data of “diffuseness” from the pathological image data is displayed in a superimposed manner on the original pathological image data. In addition, FIG. 7 illustrates an image 700 in which a heat map 701 indicating a portion (that is, a region having a feature amount with high roughness) serving as a basis of inference calculated by the observation data basis calculation unit 113 when the observation data inference unit 111 inferred observation data of “roughness” from the pathological image data is displayed in a superimposed manner on the original pathological image data. In addition, FIG. 8 illustrates an image 800 in which a heat map 801 indicating a portion (that is, a lesion suffering from the disease YYY) serving as a basis of inference calculated by the finding data basis calculation unit 114 when the finding data inference unit 112 infers the finding data having the disease name “YYY” from the pathological image data is displayed in a superimposed manner on the original pathological image data.

When the output result of the neural network is inverted or greatly changed when a specific input data item (feature amount) is changed, LIME estimates the item as “high importance in determination”. For example, each of the basis calculation units 113 and 114 generates another model (basis model) that is locally approximated in order to indicate the reason (basis) of the inference in the machine learning model used by the corresponding inference units 111 and 112. Each of the basis calculation units 113 and 114 generates a locally approximate basis model for a combination of input information (pathological image data) and an output result corresponding to the input information. Then, when the observation data and the finding data are output from the corresponding inference units 111 and 112, respectively, the basis calculation units 113 and 114 can generate the basis information regarding each of the inferred observation data and finding data using the basis model, and can similarly generate the basis images as illustrated in FIGS. 6 to 8.

The TCAV is an algorithm that calculates the importance of a concept (a concept that can be easily understood by humans) for prediction of a trained model. For example, each of the basis calculation units 113 and 114 generates a plurality of pieces of input information obtained by duplicating or changing the input information (pathological image data), inputs each of the plurality of pieces of input information to a model (explanation target model) as a generation target of the basis information, and outputs a plurality of pieces of output information corresponding to each piece of input information from the explanation target model. Then, each of basis calculation units 113 and 114 learns a basis model using a combination (pair) of each of the plurality of pieces of input information and each of the plurality of pieces of corresponding output information as learning data, and generates a basis model that is locally approximated with another interpretable model for the target input information. Then, when the observation data and the finding data are output from the corresponding inference units 111 and 112, respectively, the basis calculation units 113 and 114 can generate the basis information regarding each of the observation data and the finding data using the basis model, and can similarly generate the basis images as illustrated in FIGS. 6 to 8.

Attention is a model for learning attention points. In a case where the attention model is applied to each of the basis calculation units 113 and 114, for example, a portion that is pathologically characteristic in the input pathological image data is extracted and a response sentence describing the portion is generated. Then, in a case where there is a response sentence approximate to the observation data output from the observation data inference unit 111, a corresponding portion in the input image serves as a basis of the observation data. Similarly, in a case where there is a response sentence approximate to the observation data output from the finding data inference unit 112, the corresponding portion in the input image serves as the basis of the finding data.

Of course, the basis calculation unit 114 may calculate the basis regarding each of the diagnostic label and the differential label in the inference unit 112 on the basis of an algorithm other than the above-described Grad-Cam, LIME/SHAP, TCAV, and attention models.

### F. Reliability Calculation

In this item F, a method of calculating the reliability of the output label in the observation data inference unit 111 and the finding data inference unit 112 will be described.

Several methods for calculating a reliability score of an output label in a neural network model are known. Here, three types of reliability score calculation methods (1) to (3) will be described.

(1) Neural Network Model Learned to Estimate Error of Output

As illustrated in FIG. 9, in the neural network model, learning is performed such that the error of the output is output as the reliability score together with the original output label. The illustrated neural network corresponds to, for example, the image classification units 530 and 540 in the subsequent stage portions of the observation data inference unit 111 and the finding data inference unit 112 illustrated in FIG. 5, and is learned to output the reliability score together with the output label (observation data and finding data).

(2) Method Using Bayesian Inference

Bayesian deep learning (see, for example, Non Patent Document 2) determines uncertainty of an inference result using a dropout (random invalidation of some model parameters) not only at the time of learning but also at the time of inference. When data (pathological image data) is input to the machine learning model used in each of the observation data inference unit 111 and the finding data inference unit 112, the data passes through the neuron missing due to the dropout and an output label characterized by the weight of the path is obtained. However, even if the same data is input, the data passes through different paths and is output, so that the output is dispersed. A large variance of the output means that the uncertainty in the inference of the machine learning model is large, that is, the reliability is low.

(3) Method Using Prediction Probability (in Case of Classification Problem)

In the case of a classification problem for which prediction is obtained with a probability of 0.0 to 1.0, it can be determined that the reliability score is high in a case where a result such as 0.0 or 1.0 is obtained, 0.5 (close to 50%) in the case of binary classification, and the reliability score is low in a case where the probability of the class having the highest probability is low in the case of other class classification.

### G. Creation and Presentation of Diagnosis Report

The report creation unit 116 creates a diagnosis report of the pathological image data on the basis of the observation data and the finding data input from the observation data inference unit 111 and the finding data inference unit 112 and the respective bases calculated by the observation data basis calculation unit 113 and the finding data basis calculation unit 114.

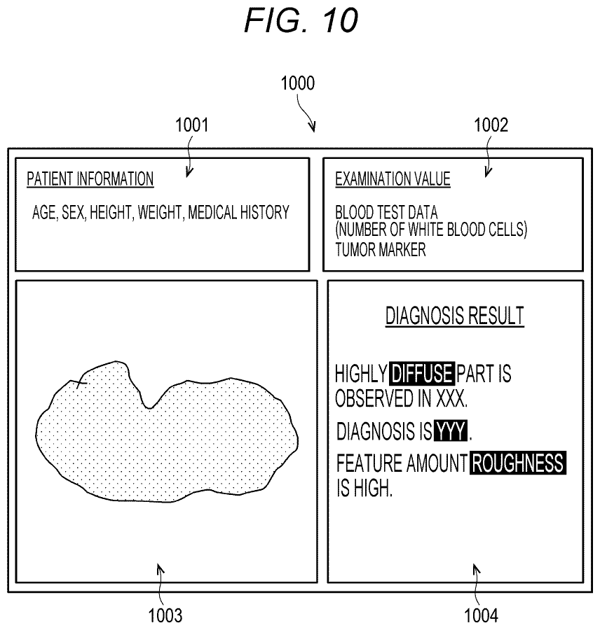

FIG. 10 illustrates a configuration example of a diagnosis report 1000 created by the report creation unit 116. Here, it is assumed that the observation data inference unit 111 outputs “diffuseness” and “roughness” as the observation data, the finding data inference unit 112 outputs the disease name “YYY” of the lesion as the finding data, and the observation data basis calculation unit 113 and the finding data basis calculation unit 114 output the calculation result of the basis of each inference. The diagnosis report 1000 illustrated in FIG. 10 is configured in the form of an electronic medical record, for example, and includes patient information 1001, an examination value 1002, pathological image data 1003, and a diagnosis result 1004. The diagnosis report 1000 is displayed, for example, on a screen of a monitor display (not illustrated).

The patient information 1001 includes information such as age, sex, and smoking history of the corresponding patient, and the examination value 1002 includes values such as blood test data and a tumor marker of the corresponding patient. The pathological image data 1003 includes an image obtained by scanning a microscopic observation image of a stained lesion placed on a glass slide. The report creation unit 116 performs natural language processing on the basis of the fragmentary words of “diffuseness”, “roughness”, and “YYY” output from the observation data inference unit 111 and the finding data inference unit 112 and the basis calculated by the observation data basis calculation unit 113 and the finding data basis calculation unit 114 to generate a fluent sentence such as “there is a highly diffuse part in XXX. The diagnosis is YYY. The feature amount roughness is high.” and include the sentence in the diagnosis report as the diagnosis result 1004. Note that the medical diagnosis system 100 may be equipped with a voice synthesis function and may read out the diagnosis result “there is a highly diffuse part in XXX. The diagnosis is YYY. The feature amount roughness is high.”

Furthermore, in the field of the diagnosis result 1004, character strings corresponding to the observation data and the finding data such as “diffuseness”, “roughness”, and “YYY” are highlighted to indicate that it is an inference result by the medical diagnosis system 100. Then, in a case where the monitor display to be used is equipped with a graphical user interface (GUI) function, when the user (pathologist) performs a predetermined selection operation (for example, a mouse over, a touch of a panel, or the like) on any highlight character string, the basis calculated by the observation data basis calculation unit 113 or the finding data basis calculation unit 114 regarding the corresponding observation data or finding data is displayed in a superimposed manner on the pathological image data 1003.

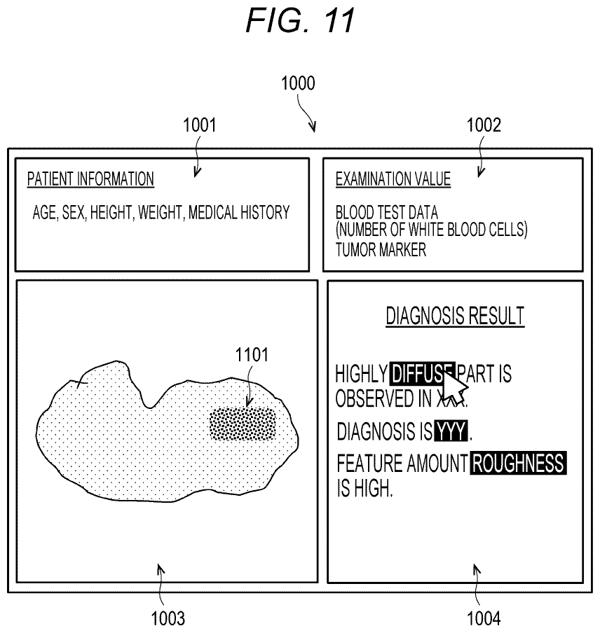

FIG. 11 illustrates a screen in which a heat map 1101 indicating a portion (that is, highly diffuse part XXX) that is a basis for inferring the observation data of “diffuseness” in response to the highlighted character string “diffuseness” being mouse-over is displayed in a superimposed manner on the pathological image data 1003.

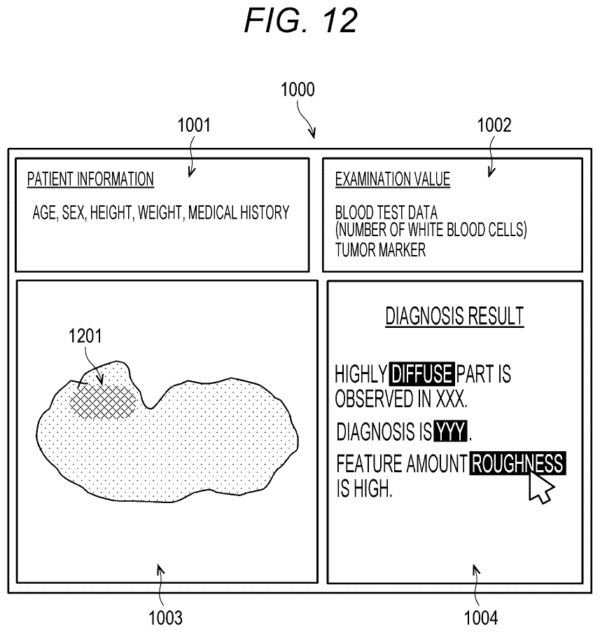

In addition, FIG. 12 illustrates a screen in which a heat map 1201 indicating a portion (that is, a region having a feature amount with high roughness) that is a basis for inferring the observation data of “diffuseness” in response to the highlighted character string “roughness” being mouse-over is displayed in a superimposed manner on the pathological image data 1003.

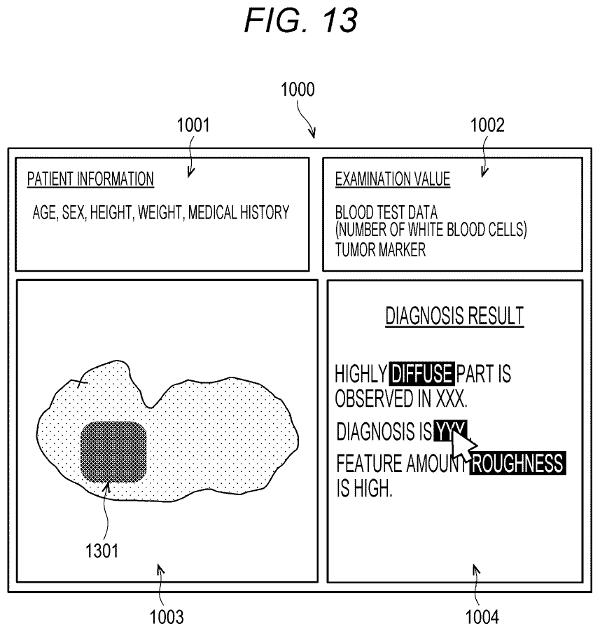

In addition, FIG. 13 illustrates a screen in which a heat map 1301 indicating a portion (that is, a lesion suffering from the disease YYY) that is a basis for inferring the finding data of the disease name “YYY” in response to the highlighted character string “diffuseness” being mouse-over is displayed in a superimposed manner on the pathological image data 1003.

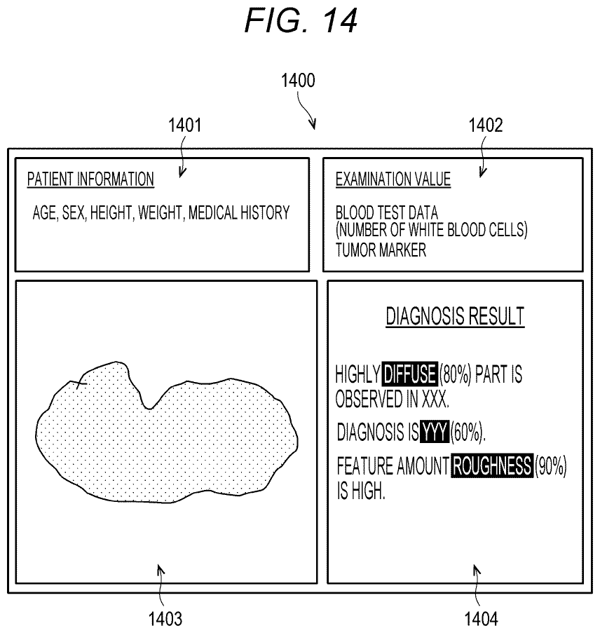

In addition, in a case where the observation data inference unit 111 and the finding data inference unit 112 output the reliability of the output label, the report creation unit 116 may create a diagnosis report displaying the reliability of each observation data and finding data. FIG. 14 illustrates a configuration example of a diagnosis report 1400 including information on the reliability of each observation data and finding data. Similarly to FIG. 10, the diagnosis report 1400 includes patient information 1401, an examination value 1402, pathological image data 1403, and a diagnosis result 1404, and is displayed on a screen of a monitor display (not illustrated), for example. Then, in the field of the diagnosis result 1404, after the character strings such as “diffuseness”, “roughness”, and “YYY” corresponding to the observation data and the finding data, each reliability is displayed in percentage.

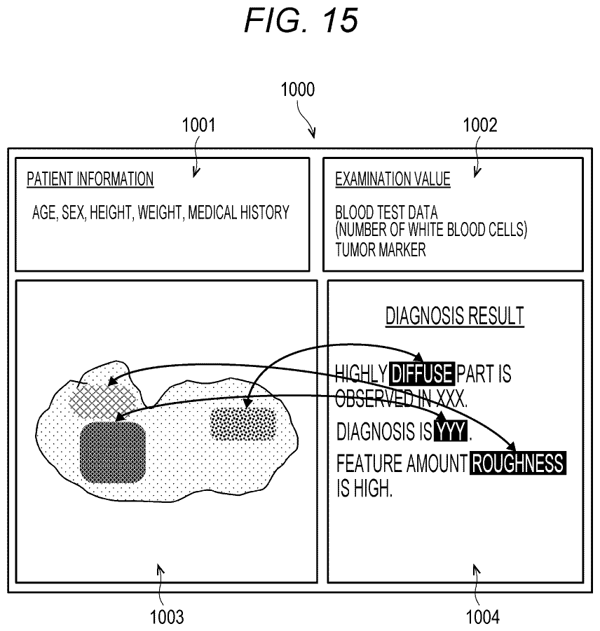

In addition, in the examples illustrated in FIGS. 10 to 14, the determination basis is displayed only for the observation data or the finding data selected by the user by an operation such as mouse over in the field of the diagnosis result 1004. However, in FIG. 15, the determination bases of all the observation data and the finding data may be simultaneously displayed. In such a case, the correspondence relationship may be clearly indicated by connecting a heat map corresponding to the observation data and the finding data with a line or the like. In addition, in a case where a plurality of heat maps serving as determination bases is simultaneously displayed, it is preferable to perform processing such as color-coding each heat map to avoid confusion.

In addition, the finding data basis calculation unit 114 may find the basis of the inferred observation data and the finding data from the observation data or the examination value instead of the pathological image (described above). In such a case, the examination value corresponding to each basis of the observation data and the finding data may be highlighted to clearly indicate the relationship between the inference result and the basis.

### H. Adoption, Modification, and Editing of Diagnosis Report

The user (pathologist) can check the diagnosis report for the pathological image data created by the medical diagnosis system 100 through the screen as illustrated in FIGS. 10 to 14. The diagnosis report includes data such as the basis of each of the observation data and the finding data, and the examination value together with a sentence of the diagnosis result including the observation data related to the features of the pathological image data and the finding data related to the diagnosis. Therefore, the user (pathologist) can make a comprehensive determination on the basis of these many pieces of information included in the diagnosis report and determine with confidence whether or not to adopt the diagnosis report created by the medical diagnosis system 100.

The user (pathologist) can input the final adoption of the diagnosis report created by the report creation unit 116 to the result adoption determination unit 117. In addition, the result adoption determination unit 117 may receive the user input and provide an editing environment such as a UI/UX in which the user (pathologist) corrects and edits the diagnosis report as well as adoption of the diagnosis report created by the report creation unit 116. In addition, the UI/UX may prepare a template or the like for supporting or simplifying the editing work of the diagnosis report. The UI/UX mentioned here may include an interaction function or may use a voice agent equipped with an artificial intelligence function. In a case where the UI/UX having an interaction function can be used, the user (pathologist) can instruct correction or editing of the diagnosis report by voice.

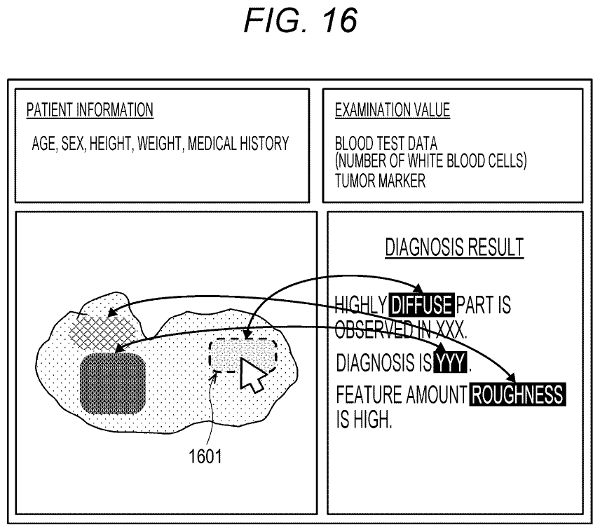

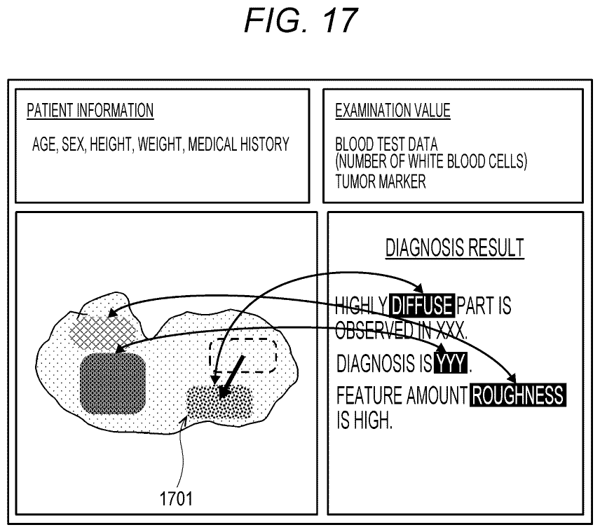

An operation example in a case where the basis of the feature data indicated in the diagnosis report is corrected will be described with reference to FIGS. 16 and 17. As illustrated in FIG. 16, in a case where the user desires to move a portion that is a basis of the feature data of “high diffuseness”, the user selects an object (heat map) 1601 indicating a range designated as the basis, for example, by a mouse click operation or the like. Then, as illustrated in FIG. 17, the user can correct the basis of the feature data by moving the object to a position 1701 appropriate to the basis of the feature data of “high diffuseness” by mouse drag operation or the like and then releasing the object. The result adoption determination unit 117 adds the diagnosis report (see FIG. 17) after correcting the basis of the feature data of “high diffuseness” to the diagnosis report DB 104 so that the diagnosis report can be utilized for subsequent relearning. In addition, the result adoption determination unit 117 may delete the diagnosis report before correction (see FIG. 16) from the diagnosis report DB 104.

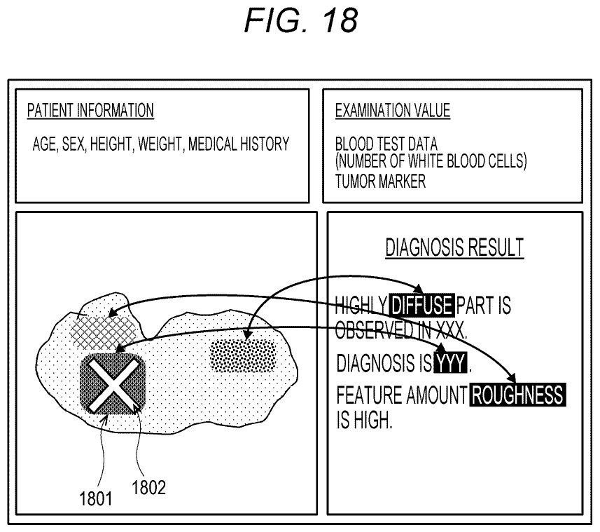

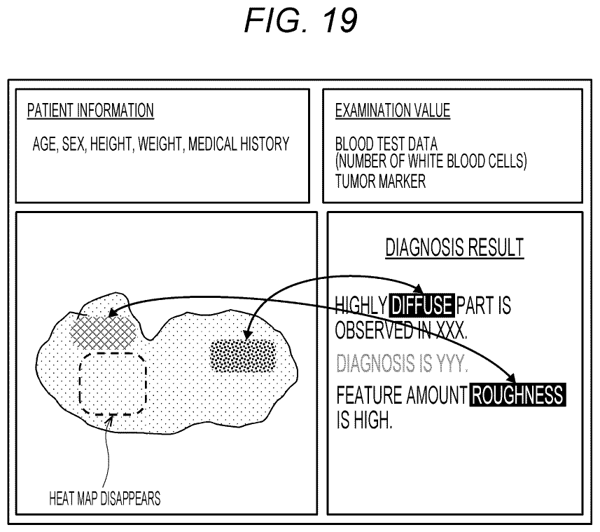

Next, an operation example in the case of correcting the finding data of the diagnosis report will be described with reference to FIGS. 18 and 19. In a case where the user (pathologist) is not satisfied with the finding data “diagnosis is YYY” on the basis indicated in the diagnosis report and desires to delete the finding data, the user instructs an object (heat map) 1801 indicating a range designated as the basis of the observation using a GUI function such as a mouse. In the example illustrated in FIG. 19, an “x” mark 1802 is attached to the designated object 1801. Then, when the deletion instruction is confirmed, the object instructed to be deleted disappears as illustrated in FIG. 19. In addition, in a case where the user is not satisfied with not only the basis of estimation but also the finding data and instructs deletion, the phrase “diagnosis is YYY.” also disappears from the column of the diagnosis result. Note that a case where the basis of the feature data is deleted instead of the finding data or a case where the feature data itself is deleted can also be realized by a similar operation to the operation illustrated in FIGS. 18 and 19.

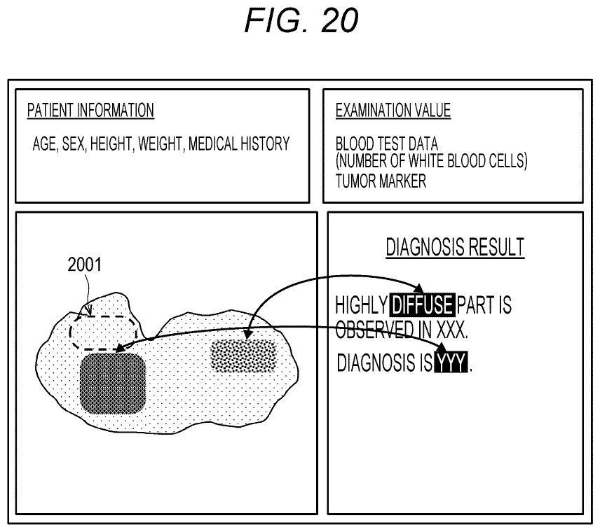

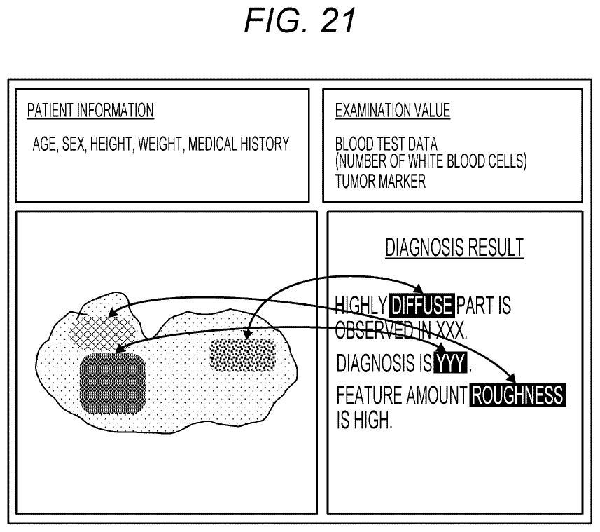

Next, an operation example in a case where the feature data and the basis thereof are added to the diagnosis report will be described with reference to FIGS. 20 and 21. Here, as illustrated in FIG. 20, it is assumed that feature data of “roughness” and an object (heat map) indicating a basis of the “roughness” are not indicated in a diagnosis report automatically generated by the medical diagnosis system 100. The user inputs observation data of “roughness” using, for example, a keyboard, a voice input function, or the like, and designates a range 2001 to be a basis of the feature data “roughness” on the pathological image data by, for example, a mouse drag operation or the like. Then, when such an instruction to add the feature data is confirmed, as illustrated in FIG. 21, the phrase “The feature amount roughness is high.” related to the added feature data is added to the diagnosis result, and the object (heat map) indicating the basis of the “roughness” appears in the designated range on the pathological image data in the pathological image data. Note that a case of addition of the finding data and the basis thereof instead of the feature data can also be realized by an operation similar to the operation illustrated in FIGS. 20 and 21.

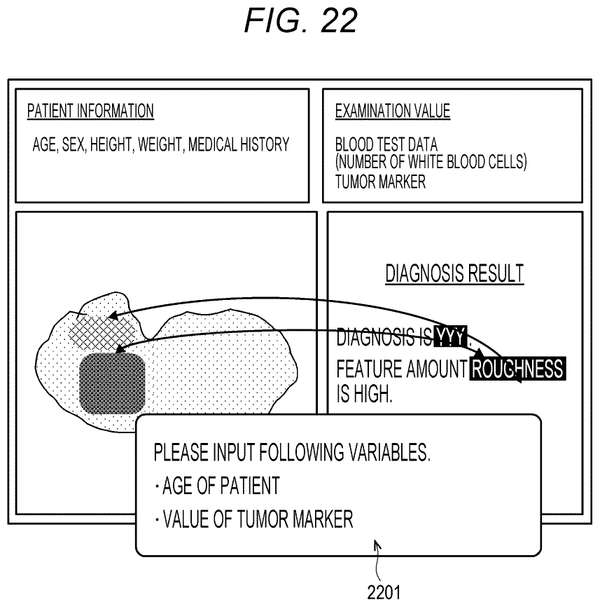

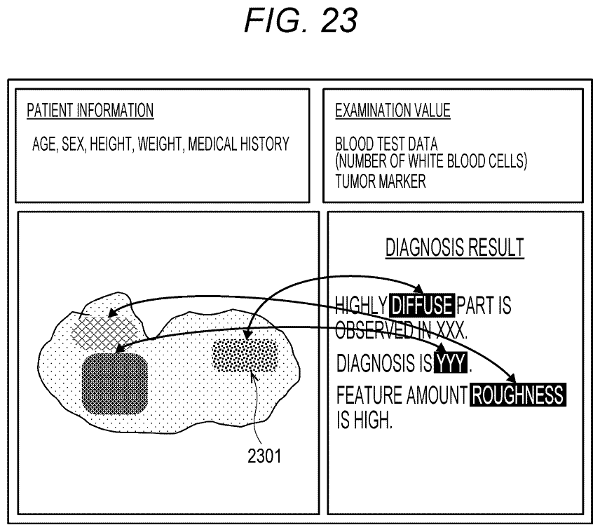

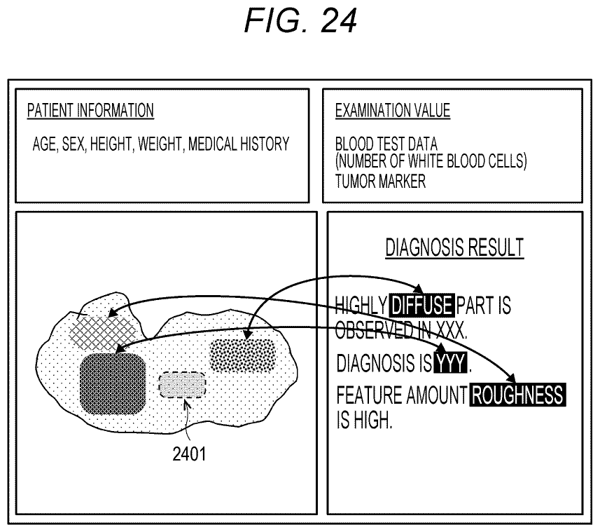

Next, an operation example in a case where a missing value detected to be missing is input will be described with reference to FIGS. 22 and 23. Here, it is assumed that, while the observation data basis calculation unit 113 is calculating the basis for inferring the observation data, the missing value detection unit 115 detects that an important variable such as age or a tumor marker is missing as the basis for inferring the feature data that is “diffuseness” by the missing value detection unit 115. The missing value detection unit 115 pops up a window 2201 prompting input of an important variable such as the age of the patient or the tumor marker on the display screen of the diagnosis report, for example. Meanwhile, the user (pathologist) inputs the missing value of the age and the tumor marker prompted for input using a keyboard, voice input, or another UI/UX. Then, when the missing value detection unit 115 inputs the missing value input by the user to the observation data inference unit 111, the observation data inference unit 111 performs inference again and outputs the observation data “diffuseness”, and the observation data basis calculation unit 113 calculates the basis of the “diffuseness”. As a result, as illustrated in FIG. 23, the phrase “a highly diffuse part is observed” representing the observation data is added to the diagnosis result, and a heat map 2301 indicating the basis of “diffuseness” appears in the pathological image data.

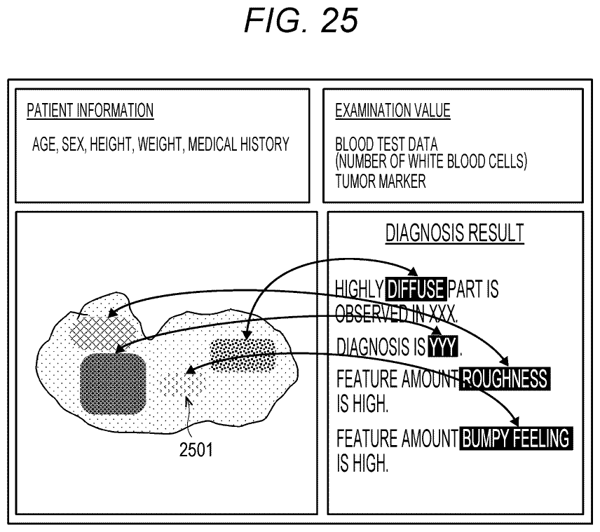

Next, an operation example in the case of assigning a name to the feature amount observed on the pathological image data will be described with reference to FIGS. 24 and 25. When finding a characteristic point 2401 on the pathological image data, the user (pathologist) designates the range 2401 by, for example, a mouse drag operation or the like, and then inputs the name “bumpy feeling” attached to the image feature amount using a keyboard, voice input, or other UI/UX. As a result, as illustrated in FIG. 24, the phrase “feature amount bumpy feeling is high” representing the observation data added to the diagnosis result is added, and a heat map 2501 indicating the basis of the “bumpy feeling” appears in a designated region on the pathological image data. Note that, also in the learning phase, that is, at the time of learning by the observation data learning unit 107, the image feature amount is named by the feature amount extraction and naming unit 109. Specifically, in a case where there is no corresponding observation data such as the “bumpy feeling” with respect to the image feature amount extracted at the time of learning of CNN, the user is prompted to name it, and the name of the “bumpy feeling” input by the user is held as the observation data.

Then, the result adoption determination unit 117 stores the diagnosis report finally adopted by the user (pathologist) through the editing operation as illustrated in FIGS. 16 to 25 in the diagnosis report DB 104 and uses the diagnosis report as learning data in the subsequent learning processing. In addition, the diagnosis report finally adopted by the user's own correction or editing is also stored in the diagnosis report DB 104 and used as learning data in the subsequent learning processing. Meanwhile, the result adoption determination unit 117 discards the diagnosis report not adopted by the user and does not add the diagnosis report to the diagnosis report DB 104.

### I. Operation in Learning Phase

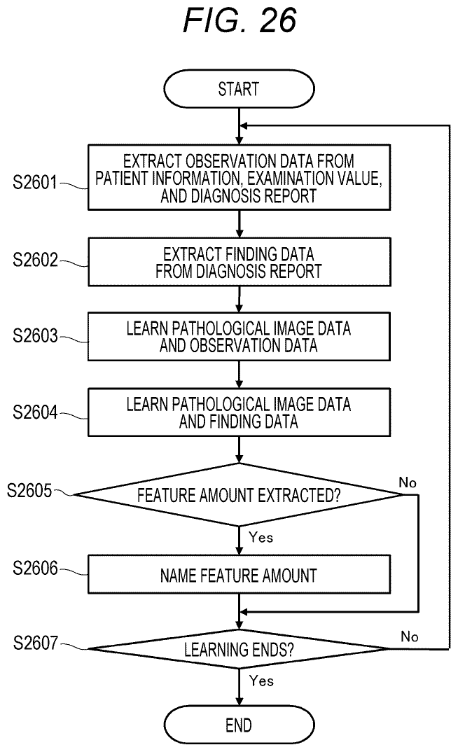

FIG. 26 illustrates a processing operation in the learning phase of the medical diagnosis system 100 in the form of a flowchart.

First, the observation data extraction unit 105 extracts observation data from the patient information, the examination value, and the diagnosis report extracted from each of the patient information DB 102, the examination value DB 103, and the diagnosis report DB 104 (step S2601). In addition, the finding data extraction unit extracts finding data from the diagnosis report extracted from the diagnosis report DB 104 (step S2602).

Then, the observation data learning unit 107 performs learning processing of the machine learning model for observation data inference by using the pathological image data extracted from the pathological image data DB 101 and the observation data extracted by the observation data extraction unit 105 (step S2603). In addition, the finding data learning unit 108 performs learning processing of the machine learning model for finding data inference by using the pathological image data extracted from the pathological image data DB 101 and the observation data extracted by the finding data extraction unit 106 (step S2604).

In a case where the CNN has extracted an image feature amount from the pathological image data in the process of learning processing of the machine learning model for observation data inference (Yes in step S2605), the feature amount extraction unit and the naming unit 109 prompts the user (pathologist) to name the feature amount, and give the feature data of the name given by the user to the feature amount (step S2606).

Then, it is checked whether or not the learning processing of the observation data learning unit 107 and the finding data learning unit 108 is completed (step S2607). For example, the Bayesian network may be used to estimate whether or not the target machine learning model has reached the limit of learning, and it may be determined whether or not the learning processing has ended. If the learning processing has not been completed (No in step S2607), the process returns to step S2601 to continue the above learning processing. In addition, when the learning processing is finished (Yes in step S2607), this processing is finished.

### J. Operation in Inference Phase

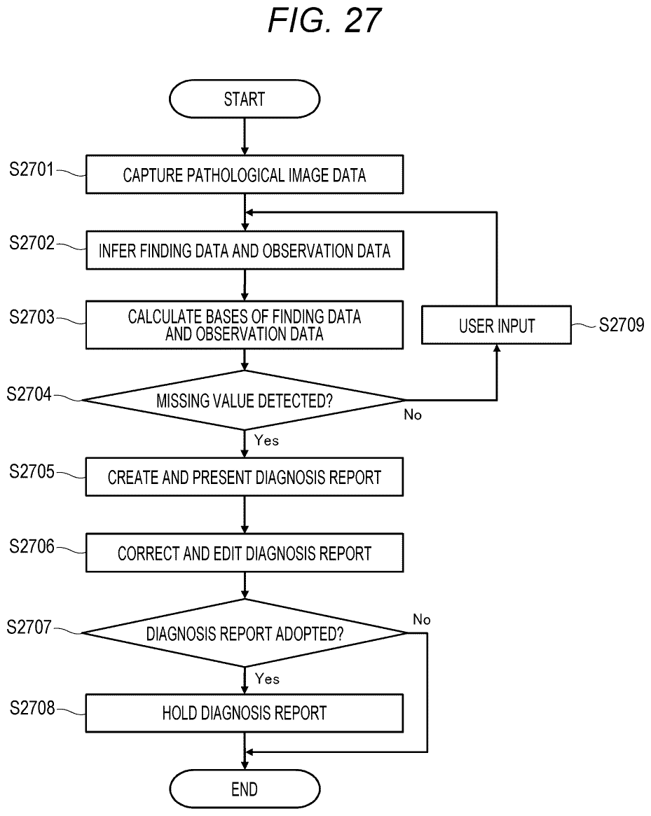

FIG. 27 illustrates a processing operation in the inference phase of the medical diagnosis system 100 in the form of a flowchart.

First, when the target pathological image data is captured (step S2701), the observation data inference unit 111 infers the observation data from the pathological image data using the machine learning model learned by the observation data learning unit 107, and the finding data inference unit 112 infers the finding data from the pathological image data using the machine learning model learned by the finding data learning unit 108 (step S2702).

Next, the observation data basis calculation unit 113 calculates the basis of the observation data inferred by the observation data learning unit 107, and the finding data basis calculation unit 114 calculates the basis of the finding data inferred by the finding data learning unit 108 (step S2703).

Here, in a case where the missing value detection unit 115 detects a missing value for the basis of the observation data calculated by the observation data basis calculation unit 113 (Yes in step S2704), the missing value detection unit prompts the user to input the missing value (step S2709), and returns to step S2702 to perform inference of the observation data and calculation of the basis of the observation data again using the missing value input by the user.

Next, the report creation unit 116 creates a diagnosis report of the pathological image data on the basis of the observation data and the finding data input from the observation data inference unit 111 and the finding data inference unit 112 and the respective bases calculated by the observation data basis calculation unit 113 and the finding data basis calculation unit 114, and presents the created diagnosis report to the user (pathologist) on the screen of the monitor display, for example (step S2705).

When the user (pathologist) checks the diagnosis report automatically created by the medical diagnosis system 100 on the screen of the monitor display or the like, the user corrects or edits the diagnosis report using the UI/UX or the like (step S2706). The correction/editing work of the diagnosis report is as described above with reference to FIGS. 16 to 25, and a detailed description thereof will be omitted here.

Then, the user (pathologist) inputs the final adoption of the diagnosis report created by the report creation unit 116 to the result adoption determination unit 117. The result adoption determination unit 117 receives an input from the user, stores the adopted diagnosis report in the diagnosis report DB 104 (step S2708), and ends this processing. By storing the adopted diagnosis report in the diagnosis report DB 104, the diagnosis report can be used as learning data in the subsequent learning processing. On the other hand, in a case where the user has not adopted the diagnosis report (step S2707), the result adoption determination unit 117 discards the diagnosis report and ends this processing.

### K. Modifications

In this item K, modifications of the medical diagnosis system 100 illustrated in FIG. 1 will be described.

### K-1. First Modification

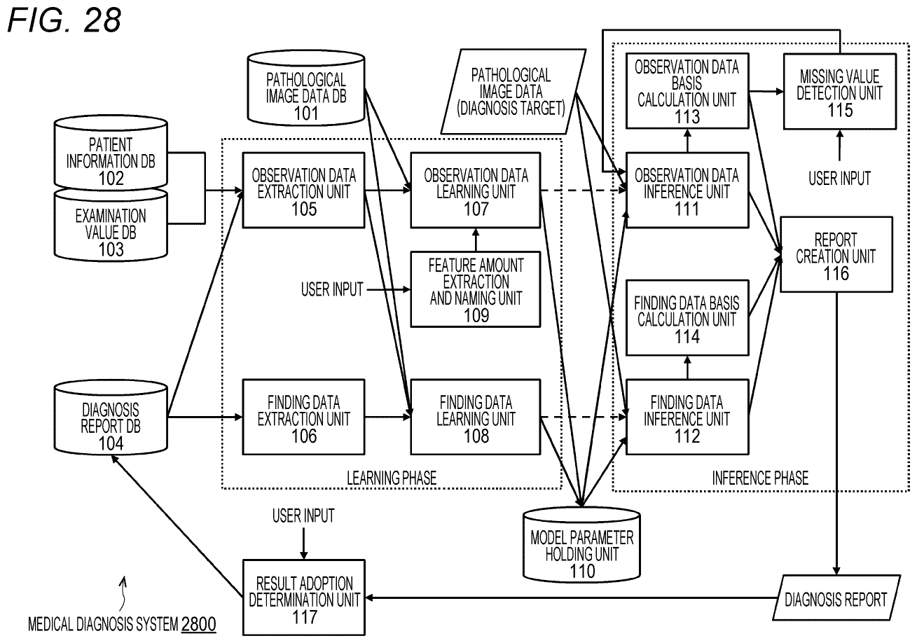

FIG. 28 schematically illustrates a functional configuration example of a medical diagnosis system 2800 according to a first modification. The same components as those of the medical diagnosis system 100 illustrated in FIG. 1 are denoted by the same reference numerals.

In the medical diagnosis system 100, the finding data learning unit 108 performs learning processing of the second machine learning model using the pathological image data as an explanatory variable and the finding data as an objective variable. On the other hand, in the medical diagnosis system 2800, the finding data learning unit 108 is configured to perform learning processing of the second machine learning model so as to infer the finding data using the pathological image data and the observation data, that is, all the variables as explanatory variables. Therefore, the medical diagnosis system 2800 according to the first modification has a feature that learning can be performed so that a diagnosis report can be output on the basis of all observation values.

The other components are similar to those of the medical diagnosis system 100 illustrated in FIG. 1, and thus, detailed description thereof is omitted here.

### K-2. Second Modification

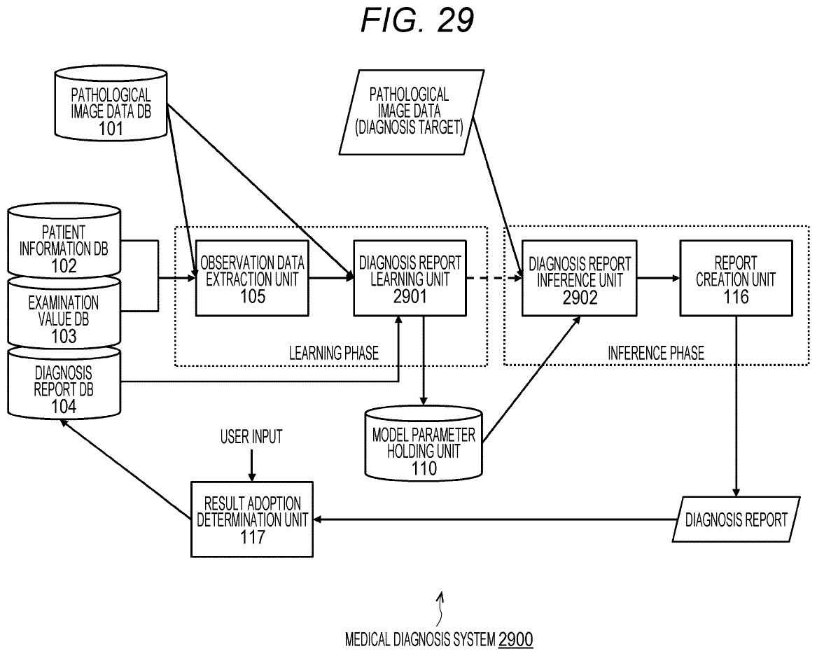

FIG. 29 schematically illustrates a functional configuration example of a medical diagnosis system 2900 according to a second modification. The same components as those of the medical diagnosis system 100 illustrated in FIG. 1 are denoted by the same reference numerals.

The medical diagnosis system 100 is configured to learn for observation data and finding data separately using two machine learning models of a machine learning model for inferring finding data from pathological image data and a machine learning model for inferring observation data from pathological image data. On the other hand, the medical diagnosis system 2900 is configured to learn only one machine learning model having the pathological image data and the observation data as explanatory variables and the diagnosis report as an objective variable, and output the diagnosis report from the pathological image data and the observation data using the machine learning model.

The medical diagnosis system 2900 includes a pathological image data DB 101 in which each data serving as an explanatory variable is converted into a database (DB), a patient information DB 102, an examination value DB 103, and a diagnosis report DB 104. It is assumed that the pathological image data, the patient information and the examination value of the corresponding patient, and the diagnosis report for the pathological image data are associated with each other.

The observation data extraction unit 105 reads the pathological image data and the patient information and the examination value corresponding to the pathological image data from each database of the pathological image data DB 101, the patient information DB 102, the examination value DB 103, and the diagnosis report DB 104, and extracts the observation data representing the pathological feature amount from the diagnosis report (text data). The observation data extraction unit 105 may perform observation data extraction processing using a machine learning model learned to detect a word or a phrase representing a feature of an image from text data.

In the learning phase, a diagnosis report learning unit 2901 performs learning processing of a machine learning model using the pathological image data and the observation data as explanatory variables and using the diagnosis report as an objective variable. Specifically, the diagnosis report learning unit 2901 performs learning processing of the machine learning model so as to infer the diagnosis report from the observation data on the basis of the learning data of a data set in which the observation data output from the observation data extraction unit 105 is input data and the diagnosis report corresponding to the pathological image data input to the observation data extraction unit 105 is a correct answer label. The machine learning model includes, for example, a neural network having a structure that mimics human neurons. The diagnosis report learning unit 2901 calculates a loss function based on an error between a label output from the machine learning model being learned to the input data and a correct answer label, and performs learning processing of the machine learning model so as to minimize the loss function.

The diagnosis report learning unit 2901 performs learning processing of the machine learning model by updating the model parameters so as to output the correct answer label for the input observation data. Then, the diagnosis report learning unit 2901 stores the model parameters of the machine learning model obtained as the learning result in the model parameter holding unit 110.

In the inference phase, the diagnosis report inference unit 2902 reads the model parameters of the machine learning model learned by the diagnosis report learning unit 2901 from the model parameter holding unit 110, and makes the machine learning model with the pathological image data and the observation data as explanatory variables and the diagnosis report as an objective variable available. Then, the diagnosis report inference unit 2902 inputs the pathological image data of the diagnosis target captured from the image capturing unit (not illustrated) and the observation data thereof to infer the diagnosis report. However, instead of directly outputting the diagnosis report, the diagnosis report inference unit 2902 may output observation data and finding data constituting the diagnosis result, and the report creation unit 116 may separately shape the diagnosis report on the basis of the observation data and the finding data. The report creation unit 116 may create a diagnosis report constituted by natural language (fluent sentence) from observation data and finding data constituted by fragmentary words and phrases using a language model such as the GPT-3, for example.

Note that, although not illustrated, the medical diagnosis system 2900 may further include a diagnosis report basis calculation unit that calculates a basis of inference of the diagnosis report.

The diagnosis report output from the diagnosis report inference unit 2902 is displayed on a screen of a monitor display (not illustrated), for example. A user (such as a pathologist) can check the contents of the diagnosis report via the display screen of the monitor display. The configuration of the display screen of the diagnosis report is as illustrated in FIG. 10, for example. In addition, the user (pathologist or the like) can appropriately browse the basis of the diagnosis report and perform an editing operation. In addition, in a case where the diagnosis report inference unit 2902 outputs the reliability of the output label, the reliability of each of the observation data and the finding data on the diagnosis report may be presented together. The editing operation of the diagnosis report may be as described in the above item H, for example.

The user (pathologist) can input the final adoption of the diagnosis report output from the diagnosis report inference unit 2902 to the result adoption determination unit 117. Then, the result adoption determination unit 117 receives an input from the user, stores the adopted diagnosis report in the diagnosis report DB 104, and uses the diagnosis report as learning data in the subsequent learning processing. In addition, the result adoption determination unit 117 discards the diagnosis report not adopted by the user and does not add the diagnosis report to the diagnosis report DB 104.

As described above, the medical diagnosis system 2900 according to the second modification can learn the diagnosis report using a single machine learning model and create the diagnosis report from the pathological image data and the observation data.

### L. Configuration Example of Information Processing Device

In this item K, an information processing device capable of realizing one or both of the learning phase and the inference phase in the medical diagnosis system 100 will be described.

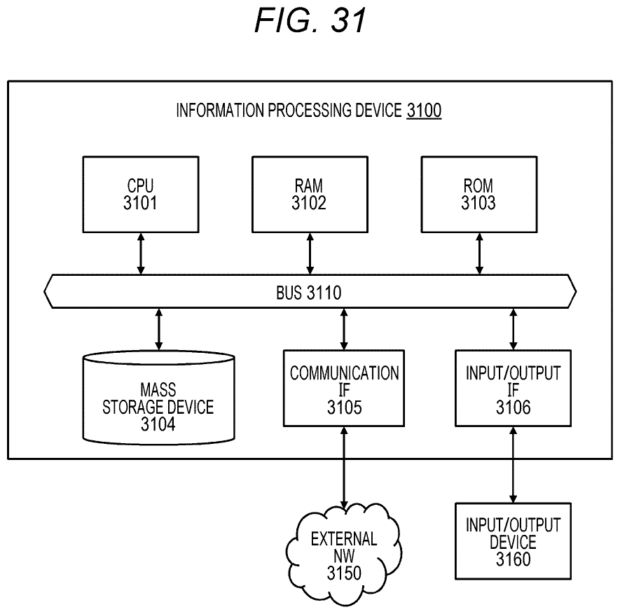

FIG. 31 illustrates a configuration example of the information processing device 3100. The information processing device 3100 includes a central processing unit (CPU) 3101, a random access memory (RAM) 3102, a read only memory (ROM) 3103, a mass storage device 3104, a communication interface (IF) 3105, and an input/output interface (IF) 3106. Each unit of the information processing device 3100 is interconnected by a bus 3110. The information processing device 3100 is configured using, for example, a personal computer.

The CPU 3101 operates on the basis of a program stored in the ROM 3103 or the mass storage device 3104, and controls the operation of each unit. For example, the CPU 3101 develops and executes various programs stored in the ROM 3103 or the mass storage device 3104 on the RAM 3102, and temporarily stores work data during program execution in the RAM 3102.

The ROM 3103 stores, in a nonvolatile manner, a boot program executed by the CPU 3101 at the time of activation of the information processing device 3100, a program depending on hardware of the information processing device 3100 such as a basic input output system (BIOS), data, and the like.

The mass storage device 3104 includes a computer-readable recording medium such as a hard disk drive (HDD) or a solid state drive (SSD). The mass storage device 3104 non-volatilely records a program executed by the CPU 3101, data used by the program, and the like in a file format. Specifically, in the medical diagnosis system 100 illustrated in FIG. 1, the mass storage device 3104 records, in a file format, a program for implementing a processing operation in which the observation data extraction unit 105 and the finding data extraction unit 106 extract the observation data and the finding data, a program for implementing a processing operation in which the observation data learning unit 107 and the finding data learning unit 108 each learn a machine learning model, model parameters (weighting coefficients of neurons and the like) of each machine learning model learned by the observation data learning unit 107 and the finding data learning unit 108, a program for implementing a processing operation in which the observation data inference unit 111 and the finding data inference unit 112 each use a learned machine learning model to infer the observation data and the finding data from the pathological image data, a program for implementing a processing operation in which the observation data basis calculation unit 113 and the finding data basis calculation unit 114 calculate the basis of inference of the observation data inference unit 111 and the finding data inference unit 112, a program for implementing a processing operation for automatically creating a diagnosis report on the basis of the inferred observation data and finding data and the calculated basis of inference, a program for implementing a processing operation for presenting a diagnosis report and performing correction and editing by a user, and the like.

The communication interface 3105 is an interface for the information processing device 3100 to connect to an external network 3150 (for example, the Internet). For example, the CPU 3101 receives data from another device or transmits data generated by the CPU 3101 to another device via the communication interface 3105.

The input/output interface 3106 is an interface for connecting an input/output device 3160 to the information processing device 1000. For example, the CPU 3101 receives data from a UI/UX device (not illustrated) including an input device such as a keyboard and a mouse via the input/output interface 3106. In addition, the CPU 3101 transmits data to an output device (not illustrated) such as a display, a speaker, or a printer via the input/output interface 3106. Furthermore, the input/output interface 3106 may function as a media interface that reads a file such as a program or data recorded in a predetermined recording medium (medium). The medium mentioned here includes, for example, an optical recording medium such as a digital versatile disc (DVD) or a phase change rewritable disk (PD), a magneto-optical recording medium such as a magneto-optical disk (MO), a tape medium, a magnetic recording medium, a semiconductor memory, or the like.

For example, in a case where the information processing device 3100 functions as the medical diagnosis system 100 in the learning phase and the inference phase, the CPU 3101 executes the program loaded on the RAM 3102 to implement the functions of the observation data extraction unit 105, the finding data extraction unit, the observation data learning unit 107, the finding data learning unit 108, the feature amount extraction and naming unit 109, the observation data inference unit 111, the finding data inference unit 112, the observation data basis calculation unit 113, the finding data basis calculation unit 114, the missing value detection unit 115, the report creation unit 116, and the result adoption determination unit 117. In addition, the mass storage device 3104 stores a program for implementing a processing operation for the observation data learning unit 107 and the finding data learning unit 108 to learn a machine learning model, model parameters (weighting coefficients of neurons and the like) of a learned machine learning model, a program for implementing a processing operation for the observation data inference unit 111 and the finding data inference unit 112 to infer the observation data and the finding data from the pathological image data using the learned machine learning models, a program for implementing a processing operation for the report creation unit 116 to automatically create a diagnosis report, and a program for the result adoption determination unit 117 to implement correction and editing of the diagnosis program and a processing operation associated with adoption by the user. Note that the CPU 3101 reads and executes files such as programs and data from the mass storage device 3104, but as another example, the programs and data may be acquired from another device (not illustrated) or data may be transferred to another device via the external network 3150.

### M. Microscope System

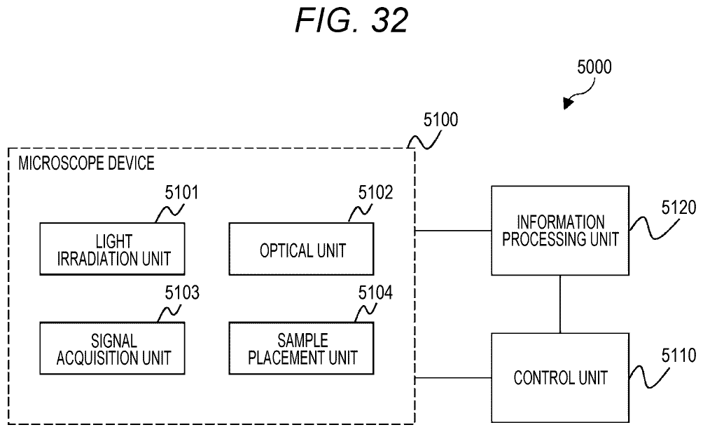

A configuration example of the microscope system of the present disclosure is illustrated in FIG. 32. A microscope system 5000 illustrated in FIG. 32 includes a microscope device 5100, a control unit 5110, and an information processing unit 5120. The microscope device 5100 includes a light irradiation unit 5101, an optical unit 5102, and a signal acquisition unit 5103. The microscope device 5100 may further include a sample placement unit 5104 on which a biological sample S is placed. Note that the configuration of the microscope device is not limited to that illustrated in FIG. 12. For example, the light irradiation unit 5101 may exist outside the microscope device 5100, and for example, a light source not included in the microscope device 5100 may be used as the light irradiation unit 5101. Alternatively, the light irradiation unit 5101 may be disposed so that the sample placement unit 5104 is sandwiched between the light irradiation unit 5101 and the optical unit 5102, and may be disposed on the side at which the optical unit 5102 exists, for example. The microscope device 5100 may be configured by one or two or more of bright field observation, phase difference observation, differential interference observation, polarization observation, fluorescent observation, and dark field observation.

The microscope system 5000 may be configured as a so-called whole slide imaging (WSI) system or a digital pathology system, and may be used for pathological diagnosis. Alternatively, the microscope system 5000 may be designed as a fluorescence imaging system, or particularly, as a multiple fluorescence imaging system.

For example, the microscope system 5000 may be used to make an intraoperative pathological diagnosis or a telepathological diagnosis. In the intraoperative pathological diagnosis, the microscope device 5100 can acquire the data of the biological sample S acquired from the subject of the operation while the operation is being performed, and then transmit the data to the information processing unit 5120. In the telepathological diagnosis, the microscope device 5100 can transmit the acquired data of the biological sample S to the information processing unit 5120 located in a place away from the microscope device 5100 (such as in another room or building). Then, in these diagnoses, the information processing unit 5120 receives and outputs the data. The user of the information processing unit 5120 can perform pathological diagnosis on the basis of the output data.

(Biological Sample)

The biological sample S may be a sample containing a biological component. The biological component may be a tissue, a cell, a liquid component of the living body (blood, urine, or the like), a culture, or a living cell (a myocardial cell, a nerve cell, a fertilized egg, or the like).

The biological sample may be a solid, or may be a specimen fixed with a fixing reagent such as paraffin or a solid formed by freezing. The biological sample can be a section of the solid. A specific example of the biological sample may be a section of a biopsy sample.

The biological sample may be one that has been subjected to a treatment such as staining or labeling. The treatment may be staining for indicating the morphology of the biological component or for indicating the substance (surface antigen or the like) contained in the biological component, and can be hematoxylin-eosin (HE) staining or immunohistochemistry staining, for example. The biological sample may be one that has been subjected to the above treatment with one or more reagents, and the reagent(s) can be a fluorescent dye, a coloring reagent, a fluorescent protein, or a fluorescence-labeled antibody.

The specimen may be prepared for the purpose of pathological diagnosis, clinical examination, or the like from a specimen or a tissue sample collected from a human body. Alternatively, the specimen is not necessarily of the human body, and may be derived from an animal, a plant, or some other material. The specimen may differ in property, depending on the type of the tissue being used (such as an organ or a cell, for example), the type of the disease being examined, the attributes of the subject (such as age, gender, blood type, and race, for example), or the subject's daily habits (such as an eating habit, an exercise habit, and a smoking habit, for example). The specimen may be accompanied by identification information (bar code information, QR code (registered trademark) information, or the like) for identifying each specimen, and be managed in accordance with the identification information.

(Light Irradiation Unit)

The light irradiation unit 5101 is a light source for illuminating the biological sample S, and is an optical unit that guides light emitted from the light source to a specimen. The light source can illuminate a biological sample with visible light, ultraviolet light, infrared light, or a combination thereof. The light source may be one or two or more of a halogen lamp, a laser light source, an LED lamp, a mercury lamp, and a xenon lamp. The light source in fluorescent observation may be of a plurality of types and/or wavelengths, and the types and the wavelengths may be appropriately selected by a person skilled in the art. The light irradiation unit may have a configuration of a transmissive type, a reflective type, or an epi-illumination type (a coaxial epi-illumination type or a side-illumination type).

(Optical Unit)

The optical unit 5102 is designed to guide the light from the biological sample S to the signal acquisition unit 5103. The optical unit may be designed to enable the microscope device 5100 to observe or capture an image of the biological sample S.

The optical unit 5102 may include an objective lens. The type of the objective lens may be appropriately selected by a person skilled in the art, in accordance with the observation method. The optical unit may also include a relay lens for relaying an image magnified by the objective lens to the signal acquisition unit. The optical unit may further include optical components other than the objective lens and the relay lens, and the optical components may be an eyepiece, a phase plate, a condenser lens, and the like.

The optical unit 5102 may further include a wavelength separation unit designed to separate light having a predetermined wavelength from the light from the biological sample S. The wavelength separation unit may be designed to selectively cause light having a predetermined wavelength or a predetermined wavelength range to reach the signal acquisition unit. The wavelength separation unit may include one or more of the following: a filter, a polarizing plate, a prism (Wollaston prism), and a diffraction grating that selectively pass light, for example. The optical component(s) included in the wavelength separation unit may be disposed in the optical path from the objective lens to the signal acquisition unit, for example. The wavelength separation unit is provided in the microscope device in a case where fluorescent observation is performed, or particularly, where an excitation light irradiation unit is included. The wavelength separation unit may be designed to separate fluorescence or white light from fluorescence.

(Signal Acquisition Unit)

The signal acquisition unit 5103 may be designed to receive light from the biological sample S, and convert the light into an electrical signal, or particularly, into a digital electrical signal. The signal acquisition unit may be designed to be capable of acquiring data about the biological sample S, on the basis of the electrical signal. The signal acquisition unit may be designed to be capable of acquiring data of an image (a captured image, or particularly, a still image, a time-lapse image, or a moving image) of the biological sample S, or particularly, may be designed to acquire data of an image enlarged by the optical unit. The signal acquisition unit includes one or more image sensors, CMOSs, CCDs, or the like that include a plurality of pixels arranged in one- or two-dimensional manner. The signal acquisition unit may include an image sensor for acquiring a low-resolution image and an image sensor for acquiring a high-resolution image, or may include an image sensor for sensing for AF or the like and an image sensor for outputting an image for observation or the like. In addition to the plurality of pixels, the imaging element can include a signal processing unit (including one, two, or three of a CPU, a DSP, and a memory) that performs signal processing using a pixel signal from each pixel, and an output control unit that controls output of image data generated from the pixel signal and processing data generated by the signal processing unit. Moreover, the imaging element can include an asynchronous event detection sensor that detects, as an event, that a luminance change of a pixel that photoelectrically converts incident light exceeds a predetermined threshold. The image sensor including the plurality of pixels, the signal processing unit, and the output control unit can be preferably designed as a one-chip semiconductor device.

(Control Unit)

The control unit 5110 controls imaging being performed by the microscope device 5100. For the imaging control, the control unit 5110 can drive movement of the optical unit 5102 and/or the sample placement unit 5104, to adjust the positional relationship between the optical unit 5102 and the sample placement unit 5104. The control unit 5110 can move the optical unit 5102 and/or the sample placement unit 5104 in a direction toward or away from each other (in the optical axis direction of the objective lens, for example). The control unit 5110 may also move the optical unit and/or the sample placement unit 5104 in any direction in a plane perpendicular to the optical axis direction. For the imaging control, the control unit 5110 may control the light irradiation unit 5101 and/or the signal acquisition unit 5103.

(Sample Placement Unit)

The sample placement unit 5104 may be designed to be capable of securing the position of a biological sample on the sample placement unit 5104, and may be a so-called stage. The sample placement unit 5104 may be designed to be capable of moving the position of the biological sample in the optical axis direction of the objective lens and/or in a direction perpendicular to the optical axis direction.

(Information Processing Unit)

The information processing unit 5120 can acquire, from the microscope device 5100, data (imaging data or the like) acquired by the microscope device 5100. The information processing unit 5120 can perform image processing on the imaging data. The image processing may include color separation processing. The color separation processing can include processing of extracting data of a light component of a predetermined wavelength or wavelength range from the imaging data to generate image data, processing of removing data of a light component of a predetermined wavelength or wavelength range from the imaging data, or the like. The image processing may also include an autofluorescence separation process for separating the autofluorescence component and the dye component of a tissue section, and a fluorescence separation process for separating wavelengths between dyes having different fluorescence wavelengths from each other. The autofluorescence separation process may include a process of removing the autofluorescence component from image information about another specimen, using an autofluorescence signal extracted from one specimen of the plurality of specimens having the same or similar properties.

The information processing unit 5120 may transmit data for the imaging control to the control unit 5110, and the control unit 5110 that has received the data may control the imaging being by the microscope device 5100 in accordance with the data.

The information processing unit 5120 may be designed as an information processing device such as a general-purpose computer, and may include a CPU, RAM, and ROM. The information processing unit may be included in the housing of the microscope device 5100, or may be located outside the housing. Further, the various processes or functions to be executed by the information processing unit 5120 may be realized by a server computer or a cloud connected via a network.

The method to be implemented by the microscope device 5100 to capture an image of the biological sample S may be appropriately selected by a person skilled in the art, in accordance with the type of the biological sample, the purpose of imaging, and the like. Examples of the imaging method are described below.

One example of the imaging method is as follows. The microscope device 5100 can first identify an imaging target region. The imaging target region may be identified so as to cover the entire region in which the biological sample exists, or may be identified so as to cover the target portion (the portion in which the target tissue section, the target cell, or the target lesion exists) of the biological sample. Next, the microscope device 5100 divides the imaging target region into a plurality of divided regions of a predetermined size, and the microscope device 5100 sequentially captures images of the respective divided regions. As a result, an image of each divided region is acquired.

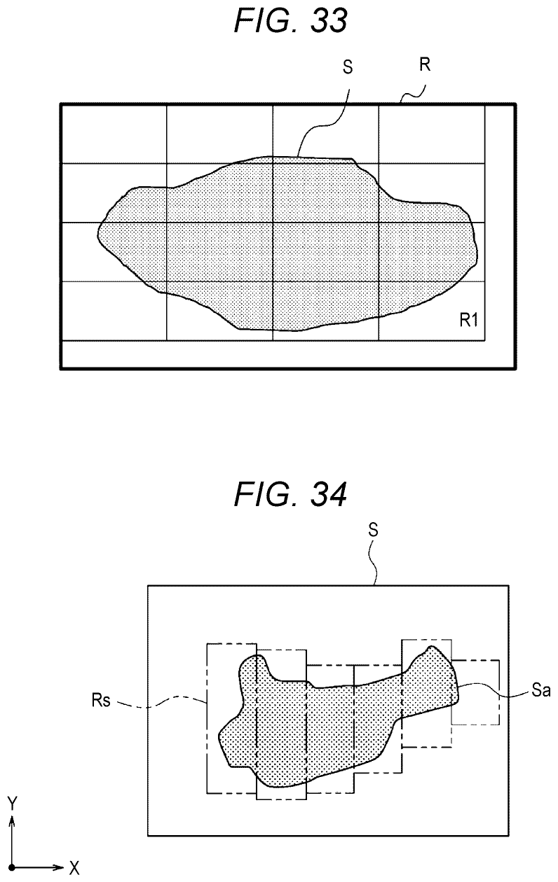

As illustrated in FIG. 33, the microscope device 5100 specifies an imaging target region R covering the entire biological sample S. The microscope device 5100 then divides the imaging target region R into 16 divided regions. The microscope device 5100 then captures an image of a divided region R1, and next captures one of the regions included in the imaging target region R, such as an image of a region adjacent to the divided region R1. After that, divided region imaging is performed until images of all the divided regions have been captured. Note that an image of a region other than the imaging target region R may also be captured on the basis of captured image information about the divided regions.

The positional relationship between the microscope device 5100 and the sample placement unit 5104 is adjusted so that an image of the next divided region is captured after one divided region is captured. The adjustment may be performed by moving the microscope device 5100, moving the sample placement unit 5104, or moving both. In this example, the imaging device that captures an image of each divided region may be a two-dimensional image sensor (an area sensor) or a one-dimensional image sensor (a line sensor). The signal acquisition unit may capture an image of each divided region via the optical unit. Further, images of the respective divided regions may be continuously captured while the microscope device 5100 and/or the sample placement unit 5104 is moved, or movement of the microscope device 5100 and/or the sample placement unit 5104 may be stopped every time an image of a divided region is captured. The imaging target region may be divided so that the respective divided regions partially overlap, or the imaging target region may be divided so that the respective divided regions do not overlap. A plurality of images of each divided region may be captured while the imaging conditions such as the focal length and/or the exposure time are changed.

Furthermore, the information processing unit 5120 can combine a plurality of adjacent divided regions to generate image data of a wider region. By performing the combining processing over the entire imaging target region, an image of a wider region can be acquired for the imaging target region. Furthermore, image data with lower resolution can be generated from the image of the divided region or the image subjected to the combining processing.

Another example of the imaging method is as follows. The microscope device 5100 can first identify an imaging target region. The imaging target region may be identified so as to cover the entire region in which the biological sample exists, or may be identified so as to cover the target portion (the portion in which the target tissue section or the target cell exists) of the biological sample. Next, the microscope device 5100 scans a region (also referred to as a “divided scan region”) of the imaging target region in one direction (also referred to as a “scan direction”) in a plane perpendicular to the optical axis, and thus captures an image. After the scanning of the divided scan region is completed, the divided scan region next to the scan region is then scanned. These scanning operations are repeated until an image of the entire imaging target region is captured.

As illustrated in FIG. 34, the microscope device 5100 specifies, as an imaging target region Sa, a region where a tissue section exists (a gray portion) in the biological sample S. The microscope device 5100 then scans a divided scan region Rs of the imaging target region Sa in the Y-axis direction. After completing the scanning of the divided scan region Rs, the microscope device 5100 then scans the divided scan region that is the next in the X-axis direction. This operation is repeated until scanning of the entire imaging target region Sa is completed.

For the scanning of each divided scan region, the positional relationship between the microscope device 5100 and the sample placement unit 5104 is adjusted so that an image of the next divided scan region is captured after an image of one divided scan region is captured. The adjustment may be performed by moving the microscope device 5100, moving the sample placement unit 5104, or moving both. In this example, the imaging device that captures an image of each divided scan region may be a one-dimensional image sensor (a line sensor) or a two-dimensional image sensor (an area sensor). The signal acquisition unit may capture an image of each divided region via a magnifying optical system. Also, images of the respective divided scan regions may be continuously captured while the microscope device 5100 and/or the sample placement unit 5104 is moved. The imaging target region may be divided so that the respective divided scan regions partially overlap, or the imaging target region may be divided so that the respective divided scan regions do not overlap. A plurality of images of each divided scan region may be captured while the imaging conditions such as the focal length and/or the exposure time are changed.

Furthermore, the information processing unit 5120 can combine a plurality of adjacent divided scan regions to generate image data of a wider region. By performing the combining processing over the entire imaging target region, an image of a wider region can be acquired for the imaging target region. Furthermore, image data with lower resolution can be generated from the image of the divided scan region or the image subjected to the combining processing.

The information processing unit 5120 is basically a device that implements the operation in the inference mode in the medical diagnosis system 100 illustrated in FIG. 1, and can be configured using the information processing device 3100 illustrated in FIG. 31. Of course, the information processing unit 5120 may also have a function of operating as a learning mode, and perform relearning or additional learning of the machine learning model to be used. The information processing unit 5120 infers a disease from the pathological image data captured by the microscope device 5100, outputs a diagnostic label and a differential label corresponding to the diagnostic label, calculates each basis of the diagnostic label and the differential label, and outputs information such as a heat map indicating each basis. Furthermore, the information processing unit 5120 includes an input device that performs user input, and receives input of final diagnosis (findings of the pathologist, for example, selection results of one of a diagnostic label and a differential label) and observation data (for example, a comment of a pathologist on a pathological image such as “high diffuseness”, and the like) by a pathologist.

The information processing unit 5120 records the pathological image data captured by the microscope device 5100 in the mass storage device 3104. In addition, the information processing unit 5120 records a diagnosis result inferred from the pathological image data, findings on the pathological image by the pathologist, and observation data in association with the pathological image data. The information processing unit 5120 may store examination values such as blood, pathological image data, findings by a pathologist, and observation data in the mass storage device 3104 for each patient in the form of an electronic medical record, for example.

### INDUSTRIAL APPLICABILITY

The present disclosure has been described in detail above with reference to the specific embodiments. However, it is obvious that those skilled in the art can make modifications and substitutions of the embodiments without departing from the gist of the present disclosure.

In the present specification, the embodiments in which the present disclosure is applied to analysis of a pathological image have been mainly described, but the gist of the present disclosure is not limited thereto. The present disclosure can be similarly applied to diagnosis of various medical images such as an X-ray image, Computed Tomography (CT), Magnetic Resonance Imaging (MRI), and an endoscopic image.

In short, the present disclosure has been described in the form of exemplification, and thus the contents described herein should not be construed in a limited manner. To determine the gist of the present disclosure, the scope of claims should be taken into consideration.

Note that the present disclosure can have the following configurations.

(1) An image diagnostic system including:
a control unit;
a diagnosis unit that estimates a diagnosis result using a machine learning model on the basis of an input image;
a diagnosis result report output unit that outputs a diagnosis result report on the basis of the diagnosis result;
a selection unit that selects a part of a diagnostic content included in the diagnostic result report; and
an extraction unit that extracts determination basis information that has affected estimation of the diagnosis content selected,
in which the control unit outputs the determination basis information.

(2) The image diagnostic system according to (1), further including
a correction unit that corrects the determination basis information,
in which the determination basis information corrected is used for relearning of the machine learning model.

(3) The image diagnostic system according to any one of (1) and (2),
in which the diagnosis unit includes an observation data inference unit that infers observation data related to a feature of the input image and a finding data inference unit that infers finding data related to diagnosis of the input image,
the control unit calculates a basis of inference of observation data by the observation data inference unit and a basis of inference of finding data by the finding data inference unit, and
the diagnosis result report output unit creates a diagnosis report of the input image on the basis of the observation data and the finding data inferred by the observation data inference unit and the finding data inference unit, respectively, and the basis calculated by the observation data basis calculation unit and the finding data basis calculation unit.

(4) The image diagnostic system according to (3),
in which the observation data inference unit infers the observation data using a first machine learning model learned with an input image as an explanatory variable and observation data as an objective variable, and
the finding data inference unit infers the finding data using a second machine learning model learned with an input image as an explanatory variable and finding data as an objective variable.

(5) The information processing device according to (4), further including:
an observation data learning unit that learns the first machine learning model using an input image as an explanatory variable and observation data extracted from patient information and an examination value corresponding to the input image as an objective variable; and
a finding data learning unit that learns the second machine learning model using an input image as an explanatory variable and finding data extracted from a diagnosis report for the input image as an objective variable.

(6) The information processing device according to (4), further including:
an observation data learning unit that learns the first machine learning model using an input image and observation data extracted from patient information and an examination value corresponding to the input image as explanatory variables; and
a finding data learning unit that learns the second machine learning model using an input image and the observation data corresponding to the input image as explanatory variables and finding data extracted from a diagnosis report for the input image as an objective variable.

(7) The information processing device according to any one of (5) or (6), further including:
an observation data extraction unit that extracts observation data from a diagnosis report for an input image on the basis of an input image, and patient information and an examination value corresponding to the input image; and
a finding data extraction unit that extracts finding data from a diagnosis report for an input image.

(8) The information processing device according to any one of (3) to (7),
in which at least one of the observation data inference unit or the finding data inference unit outputs reliability of inference, and the report creation unit creates the diagnosis report including information on the reliability.

(9) The information processing device according to any one of (3) to (8), further including
a presentation unit that presents the diagnosis report.

(10) The information processing device according to (9),
in which the presentation unit displays the basis of each inference of the observation data and the finding data in a superimposed manner on an original input image.

(11) The information processing device according to any one of (9) or (10),
in which the presentation unit displays the basis of each inference on an original input image in association with the observation data and the finding data.

(12) The information processing device according to any one of (9) to (11),
in which the presentation unit further presents reliability of each inference of the observation data and the finding data.

(13) The information processing device according to any one of (3) to (10), further including
a determination unit that determines whether or not to adopt the diagnosis report on the basis of a user input.

(14) The information processing device according to (13),
in which the determination unit performs at least one of correction of the basis of the observation data or the finding data in the diagnosis report, deletion of the observation data or the finding data, or addition of the observation data or finding data and the basis thereof on the basis of a user input.

(15) The information processing device according to any one of (3) to (14), further including
a naming unit that extracts a composite variable and names the composite variable on the basis of a user input.

(16) The information processing device according to (15),
in which the composite variable includes at least one of an image feature amount extracted at a time of learning an input image or a composite variable of observation values.

(17) The information processing device according to any one of (3) to (16), further including
a detection unit that detects a loss of importance of a variable that is a basis when the observation data basis calculation unit calculates the basis of the inference of the observation data by the observation data inference unit.

(18) The information processing device according to (17),
in which the detection unit prompts a user to input a missing value detected, and the observation data inference unit performs inference of the observation data again using the missing value input from the user.

(19) An image diagnostic system that processes information regarding an input image, the image diagnostic system including:
an observation data extraction unit that extracts observation data on the basis of an input image, and patient information and an examination value corresponding to the input image;
a learning unit that performs learning of a machine learning model with an input image and observation data related to the input image as explanatory variables and a diagnosis report of the input image as an objective variable; and
an inference unit that infers a diagnosis report from an input image, and patient information and an examination value corresponding to the input image, using the machine learning model learned.

(20) An image diagnostic method for diagnosing an input image, the image diagnostic method including:
an observation data inference step of inferring observation data related to a feature of the input image;
a finding data inference step of inferring finding data related to diagnosis of the input image;
an observation data basis calculation step of calculating a basis of inference of the observation data in the observation data inference step;
a finding data basis calculation step of calculating a basis of inference of the finding data in the finding data inference step; and
a report creation step of creating a diagnosis report of the input image on the basis of the observation data and the finding data inferred in each of the observation data inference step and the finding data inference step and the basis calculated in each of the observation data basis calculation step and the finding data basis calculation step.

(21) A computer program described in a computer readable format to execute processing of information regarding a medical image on a computer, the computer program causing the computer to function as:
an observation data inference unit that infers observation data related to a feature of the medical image;
a finding data inference unit that infers finding data related to diagnosis of the medical image;
an observation data basis calculation unit that calculates a basis of inference of the observation data by the observation data inference unit;
a finding data basis calculation unit that calculates a basis of inference of the finding data by the finding data inference unit; and
a report creation unit that creates a diagnosis report of the medical image on the basis of the observation data and the finding data inferred by the observation data inference unit and the finding data inference unit, respectively, and the basis calculated by the observation data basis calculation unit and the finding data basis calculation unit.

(22) A medical diagnosis system including:
an observation data learning unit that learns a first machine learning model having a medical image as an explanatory variable and observation data as an objective variable;
a finding data learning unit that learns a second machine learning model having a medical image as an explanatory variable and finding data as an objective variable;
an observation data inference unit that infers observation data related to a feature of a medical image using the first machine learning model learned;
a finding data inference unit that infers finding data related to diagnosis of a medical image using the second machine learning model learned;
an observation data basis calculation unit that calculates a basis of inference of the observation data by the observation data inference unit;
a finding data basis calculation unit that calculates a basis of inference of the finding data by the finding data inference unit;
a report creation unit that creates a diagnosis report of the medical image on the basis of the observation data and the finding data inferred by the observation data inference unit and the finding data inference unit, respectively, and the basis calculated by the observation data basis calculation unit and the finding data basis calculation unit;
a presentation unit that presents the diagnosis report; and
a determination unit that determines whether or not to adopt the diagnosis report on the basis of a user input.

### REFERENCE SIGNS LIST

100 Medical diagnosis system
101 Pathological image data DB
102 Patient information DB
103 Examination value DB
104 Diagnosis report DB
105 Observation data extraction unit
106 Finding data extraction unit
107 Observation data learning unit
108 Finding data learning unit
109 Feature amount extraction and naming unit
110 Model parameter holding unit
111 Observation data inference unit
112 Finding data inference unit
113 Observation data basis calculation unit
114 Finding data basis calculation unit
115 Missing value detection unit
116 Report creation unit
117 Result adoption determination unit
200 Data adjustment device
311 Influence degree evaluation unit
312 Learning state determination unit
313 Additional data generation unit
401 Generator
402 Discriminator
502, 504, 506 Convolutional layer output
503, 505 Pooling layer output
507, 517 Convolutional layer output
508, 518 Fully connected layer
509, 519 Output layer
520 Feature amount extraction unit
530, 540 Image classification unit
2800 Medical diagnosis system (first modification)
2900 Medical diagnosis system (second modification)
3100 Information processing device
3101 CPU
3102 RAM
3103 ROM
1004 Mass storage device
3105 Communication interface
3106 Input/output interface
3110 Bus
3150 External network
3160 Input/output device
5000 Microscope system
5100 Microscope device
5101 Light irradiation unit
5102 Optical unit
5103 Signal acquisition unit
5104 Sample placement unit
5110 Control unit
5120 Information processing unit

1. An image diagnostic system comprising:
a control unit;
a diagnosis unit that estimates a diagnosis result using a machine learning model on a basis of an input image;
a diagnosis result report output unit that outputs a diagnosis result report on a basis of the diagnosis result;
a selection unit that selects a part of a diagnostic content included in the diagnostic result report; and
an extraction unit that extracts determination basis information that has affected estimation of the diagnosis content selected,
wherein the control unit outputs the determination basis information.

2. The image diagnostic system according to claim 1, further comprising
a correction unit that corrects the determination basis information,
wherein the determination basis information corrected is used for relearning of the machine learning model.

3. The image diagnostic system according to claim 1,
wherein the diagnosis unit includes an observation data inference unit that infers observation data related to a feature of the input image and a finding data inference unit that infers finding data related to diagnosis of the input image,
the control unit calculates a basis of inference of observation data by the observation data inference unit and a basis of inference of finding data by the finding data inference unit, and
the diagnosis result report output unit creates a diagnosis report of the input image on a basis of the observation data and the finding data inferred by the observation data inference unit and the finding data inference unit, respectively, and the basis calculated by the observation data basis calculation unit and the finding data basis calculation unit.

4. The image diagnostic system according to claim 3,
wherein the observation data inference unit infers the observation data using a first machine learning model learned with an input image as an explanatory variable and observation data as an objective variable, and
the finding data inference unit infers the finding data using a second machine learning model learned with an input image as an explanatory variable and finding data as an objective variable.

5. The image diagnostic system according to claim 4, further comprising:
an observation data learning unit that learns the first machine learning model using an input image as an explanatory variable and observation data extracted from patient information and an examination value corresponding to the input image as an objective variable; and
a finding data learning unit that learns the second machine learning model using an input image as an explanatory variable and finding data extracted from a diagnosis report for the input image as an objective variable.

6. The image diagnostic system according to claim 4, further comprising:
an observation data learning unit that learns the first machine learning model using an input image and observation data extracted from patient information and an examination value corresponding to the input image as explanatory variables; and
a finding data learning unit that learns the second machine learning model using an input image and the observation data corresponding to the input image as explanatory variables and finding data extracted from a diagnosis report for the input image as an objective variable.

7. The image diagnostic system according to claim 5, further comprising:
an observation data extraction unit that extracts observation data from a diagnosis report for an input image on a basis of an input image, and patient information and an examination value corresponding to the input image; and
a finding data extraction unit that extracts finding data from a diagnosis report for an input image.

8. The image diagnostic system according to claim 3,
wherein at least one of the observation data inference unit or the finding data inference unit outputs reliability of inference, and the report creation unit creates the diagnosis report including information on the reliability.

9. The image diagnostic system according to claim 1, further comprising
a presentation unit that presents the diagnosis report.

10. The image diagnostic system according to claim 9,
wherein the presentation unit displays the basis of each inference of the observation data and the finding data in a superimposed manner on an original input image.

11. The image diagnostic system according to claim 9,
wherein the presentation unit displays the basis of each inference on an original input image in association with the observation data and the finding data.

12. The image diagnostic system according to claim 9,
wherein the presentation unit further presents reliability of each inference of the observation data and the finding data.

13. The image diagnostic system according to claim 3, further comprising
a determination unit that determines whether or not to adopt the diagnosis report on a basis of a user input.

14. The image diagnostic system according to claim 13,
wherein the determination unit performs at least one of correction of the basis of the observation data or the finding data in the diagnosis report, deletion of the observation data or the finding data, or addition of the observation data or finding data and the basis thereof on a basis of a user input.

15. The image diagnostic system according to claim 3, further comprising
a naming unit that extracts a composite variable and names the composite variable on a basis of a user input.

16. The image diagnostic system according to claim 15,
wherein the composite variable includes at least one of an image feature amount extracted at a time of learning an input image or a composite variable of observation values.

17. The image diagnostic system according to claim 3, further comprising
a detection unit that detects a loss of importance of a variable that is a basis when the observation data basis calculation unit calculates the basis of the inference of the observation data by the observation data inference unit.

18. The image diagnostic system according to claim 17,
wherein the detection unit prompts a user to input a missing value detected, and the observation data inference unit performs inference of the observation data again using the missing value input from the user.

19. An image diagnostic system that processes information regarding an input image, the image diagnostic system comprising:
an observation data extraction unit that extracts observation data on a basis of an input image, and patient information and an examination value corresponding to the input image;
a learning unit that performs learning of a machine learning model with an input image and observation data related to the input image as explanatory variables and a diagnosis report of the input image as an objective variable; and
an inference unit that infers a diagnosis report from an input image, and patient information and an examination value corresponding to the input image, using the machine learning model learned.

20. An image diagnostic method for diagnosing an input image, the image diagnostic method comprising:
an observation data inference step of inferring observation data related to a feature of the input image;
a finding data inference step of inferring finding data related to diagnosis of the input image;
an observation data basis calculation step of calculating a basis of inference of the observation data in the observation data inference step;
a finding data basis calculation step of calculating a basis of inference of the finding data in the finding data inference step; and
a report creation step of creating a diagnosis report of the input image on a basis of the observation data and the finding data inferred in each of the observation data inference step and the finding data inference step and the basis calculated in each of the observation data basis calculation step and the finding data basis calculation step.
